(19) United States
(12) Patent Application Publication
(10) Pub. No.: US 2011/0034389 A1
(43) Pub. Date: Feb. 10, 2011

(54) ACTIVE AGENTS, COMPOSITIONS, AND METHODS FOR INHIBITING AND REVERSING PLATELET FUNCTION

(75) Inventors: Dean Y, Li, Salt Lake City, UT (US); Matthew Smith, Salt Lake City, UT (US); Andy Weyrich, Salt Lake City, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: UNIVERSITY OF UTAH RESEARCH FOUNDATION, Salt Lake City, UT (US)

(21) Appl. No.: 12/667,119

(22) PCT Filed: Oct. 26, 2009
(86) PCT No.: PCT/US09/62107

§ 371 Date: Feb. 24, 2010

Related U.S. Application Data
(60) Provisional application No. 61/108,357, filed on Oct. 24, 2008.

(51) Int. Cl. A61K 38/17; C07K 14/47; C12N 5/078; A61P 7/02; A61P 7/00

(52) U.S. Cl. 514/13.8; 514/13.5; 514/14.9; 530/350; 435/375

(57) ABSTRACT

Active agents, compositions, and methods for inhibiting and reversing platelet function are provided herein. In particular embodiments, the active agents provided herein inhibit or reverse platelet function. In particular embodiments, the compositions described herein are pharmaceutical formulations. Methods of inhibiting and reversing platelet function are also provided herein. In particular embodiments, methods as described herein include administration of a Sema3E polypeptide. Further, methods of treating pathologic conditions associated with platelet function (i.e., platelet activation) and methods of screening active agent candidates are also provided.

### STATEMENT AS TO RIGHTS TO INVENTIONS MADE UNDER FEDERALLY SPONSORED RESEARCH OR DEVELOPMENT

This invention was made with support from the government of the United States of America under R01 HL077671 awarded by National Institutes of Health. The United States Government has certain rights to this invention

### BACKGROUND

Platelets, or thrombocytes, play a fundamental role in hemostasis, leading to the formation of blood clots. If the number of platelets is too low or if platelets become dysfunctional, excessive bleeding can occur. However, platelet activation and function are also associated with various pathologic conditions, such as stroke, heart attack, pulmonary embolism, unstable angina, angina, atrial fibrillation, and thrombosis associated with the blockage of blood vessels to other parts of the body, such as the extremities of the arms or legs (e.g., deep venous thrombosis).

Platelet function can be triggered by injury, or by one of several agonists, including thrombin, thromboxane, such as thromboxane A2, and adenosine diphosphate (“ADP”). Platelet activity is associated with one or more of platelet spreading, platelet adhesion, platelet aggregation, α-granule release, and clotting (thrombosis). Active agents and compositions capable of inhibiting platelet activity (e.g., one, more, or all of platelet spreading, adhesion, aggregation, α-granule release, and clotting) would be useful in methods for preventing or treating the serious pathologic conditions associated with platelet activity.

### SUMMARY

Active agents and compositions for inhibiting and reversing platelet function are provided herein. In particular embodiments, the active agents and compositions provided herein inhibit or reverse platelet function promoted by multiple agonists. The active agents described herein inhibit or reverse one or more platelet functions selected from platelet spreading, adhesion, aggregation, α-granule release, and clotting, and in specific embodiments the active agents include semaphorin 3E (“Sema3E”) polypeptides. For example, in specific embodiments, the active agent is selected from mammalian Sema3E polypeptides, such as a human or mouse Sema3E, as well as derivatives, homologs, and analogs of such polypeptides that inhibit or reverse platelet function. As is described herein, it has been found that Sema3E polypeptides are not only potent inhibitors of platelet function, but Sema3E polypeptides are also capable of reversing platelet function. The compositions as described herein include one or more active agents according to the present description. In particular embodiments, the compositions described herein are pharmaceutical formulations.

Methods of inhibiting or reversing platelet function are also provided herein. In specific embodiments, the methods include administering an active agent, wherein administering such active agent results in inhibition or reversal of platelet activity. In one such embodiment, the active agent administered is an active agent as described herein and administration of the active agent results in inhibition or reversal of one or more platelet functions selected from platelet spreading, adhesion, aggregation, α-granule release, and clotting. In another such embodiment, administering an active agent as described herein inhibits activation of Rap1b, resulting in inhibition of activation of α_{IIb}β₃ integrin. In yet another embodiment, administering an active agent as described herein promotes activation of one or more plexin receptors, resulting in inhibition or reversal of one or more platelet functions as described herein.

In each embodiment, where an active agent is administered to inhibit or reverse platelet function, such active agent can be an active agent as described herein and, if desired, can be administered in a composition as described herein, such as a pharmaceutical formulation. Therefore, in specific embodiments, the methods for inhibiting or reversing platelet function described herein include administering an active agent selected from mammalian Sema3E polypeptides, such as a human or mouse Sema3E, as well as derivatives, homologs, and analogs of such polypeptides that inhibit activation of Rap1b. In additional embodiments, the methods for inhibiting or reversing platelet function described herein include administering an active agent selected from mammalian Sema3E polypeptides, such as a human or mouse Sema3E, as well as derivatives, homologs, and analogs of such polypeptides, that bind to and are biologically active at one or more plexin receptors.

Methods of treating pathologic conditions associated with platelet function are also described herein. A pathologic condition treated by the methods described herein may be selected from pathologic conditions associated with one or more of platelet spreading, adhesion, aggregation, α-granule release, and clotting. Specific examples of such conditions include, but are not limited to, stroke, myocardial infarction, unstable angina, angina, thrombosis, including deep venous thrombosis, pulmonary embolism, and atrial fibrillation. In one embodiment, a method of treatment as described herein, includes administering a therapeutically effective amount of an active agent as described herein to a patient suffering from a pathologic condition resulting from one or more of platelet spreading, adhesion, aggregation, α-granule release or clotting. In one such embodiment, the method includes administering a therapeutically effective amount of an active agent as described herein to a patient suffering from a condition selected from stroke, myocardial infarction, unstable angina, angina, thrombosis, including deep venous thrombosis, pulmonary embolism, and atrial fibrillation. The active agent administered in the methods described herein may be a polypeptide active agent, such as, for example, a Sema3E polypeptide. For example, in one embodiment, a method as described herein includes administering a mammalian Sema3E polypeptide selected from a human or mouse Sema3E polypeptide, as well as derivatives, homologs, and analogs of such polypeptides, wherein the polypeptide administered inhibits or reverses one or more platelet function.

In certain embodiments, the methods for treating a pathologic condition associated with platelet function include inhibiting activation of Rap1b. Rap1b activation is a common feature of platelet activity. In particular it is believed that activation of Rap1b is necessary for activation of α_{IIb}β₃ integrin. In one such embodiment, therefore, a method for treating a pathologic condition associated with platelet function includes inhibiting activation of Rap1b activation such that activation of α_{IIb}β₃ integrin is inhibited. In specific embodiments, the methods for treating a pathologic condition associated with platelet function by inhibition of Rap1b include administering a Sema3E polypeptide as described herein. For example, in one such embodiment, the method includes administering a therapeutically effective amount of a mammalian Sema3E polypeptide, such as a human or mouse Sema3E, including derivatives, homologs, and analogs of such polypeptides, wherein the polypeptide administered inhibits activation of Rap1b. The pathologic condition treated by a method involving inhibition of activation of Rap1b can be selected from conditions resulting from or associated with one or more of platelet spreading, adhesion, aggregation, α-granule release or clotting, including, by way of example and not limitation, stroke, myocardial infarction, unstable angina, angina, thrombosis, including deep venous thrombosis, pulmonary embolism, and atrial fibrillation.

In yet another embodiment, a method of treating a pathologic condition associated with platelet function as described herein includes administering an active agent that activates one or more plexin receptors. In one such embodiment, therefore, a method for treating a pathologic condition associated with platelet function includes administering a Sema3E polypeptide that binds to and is biologically active at one or more plexin receptors, such as Plexin D1 or a receptor selected from the Plexin A family of receptors. In one such embodiment, the active agent for administration may be selected from a mammalian Sema3E polypeptide, such as a human or mouse Sema3E, including derivatives, homologs, and analogs of such polypeptides, wherein the polypeptide administered binds to and is biologically active at one or more plexin receptors. The pathologic condition treated by a method involving administration of an active agent that is biologically active at one or more plexin receptors can be selected from conditions resulting from or associated with one or more of platelet spreading, adhesion, aggregation, α-granule release or clotting, including, by way of example and not limitation, stroke, myocardial infarction, unstable angina, angina, thrombosis, including deep venous thrombosis, pulmonary embolism, and atrial fibrillation.

Methods of screening for compositions capable of modulating platelet function are also described herein. In particular embodiments, the screening methods described herein included evaluating the ability of one or more active agents or compositions to activate one or more plexin receptors, such as PlexinD1 or inhibit activation of Rap1b.

### BRIEF DESCRIPTION OF THE DRAWINGS

The accompanying drawings, which are incorporated in and constitute a part of this specification, illustrate several embodiments of the disclosed active agents, compositions, and method and, together with the description, serve to explain the principles of the disclosed method and compositions.

FIG. 1 illustrates that PlexinD1 is expressed at the cellular membrane of platelets. As can be seen in panel B of FIG. 1, CHO-PD1 cells, which express the PlexinD1 receptor, exhibited anti-PlexinD1 staining at the cell membrane. As is shown in panels C and D of FIG. 1, Anti-PlexinD1 staining was observed at the cell membrane in both human and mouse platelets.

FIG. 2 provides images that illustrate the differences in potency observed with different semaphorin proteins. Panel A of FIG. 2 provides an image that is illustrative of the results achieved with human platelet cells incubated with a vehicle only. Panels B, E, and F, provide images that are illustrative of the results achieved with human platelet cells incubated with Netrin 1, Slit2, and Netrin 4, respectively. Panels C, D, and G, provide images illustrative of the results achieved with human platelet cells incubated with Sema3A, Sema3F, and Sema3E polypeptides, respectively. As can be appreciated from FIG. 2, the effect of the Sema3E polypeptide was particularly potent. Panel H provides an image illustrating the effect of Sema3E is reversible, as removal and washing of the Sema3E containing media restored the platelets' ability to spread.

FIG. 3 provides images illustrating that the activity of Sema3E is preserved in mouse platelets. In particular, Panel A provides an image of mouse platelets incubated on borosilicate chamber slides coated with fibrinogen in the presence of a vehicle only, while Panel B provides an image of mouse platelets incubated on borosilicate chamber slides coated with fibrinogen in the presence of Sema3E.

FIG. 4 provides images illustrating that the activity of Sema3E is not agonist dependent. Panel A provides an image of platelets incubated with ADP in the presence of a vehicle only, while Panel B provides an image of mouse platelets incubated with ADP in the presence of Sema3E.

FIG. 5 provides images illustrating that Sema3E inhibits platelet attachment and spreading on β₃ and β₁ integrin-dependent substrates. Human platelets were incubated with a vehicle only or with media containing hSema3E and then placed on chamber slides coated with the designated substrates in the presence of thrombin. As can be appreciated by comparison between to the panels associated with cells incubated with hSema3E and cells incubated with vehicle only, Sema3E works to inhibit thrombin-induced platelet spreading and attachment on each of the substrates.

FIG. 6 provides images illustrating that Sema3E/PlexinD1 signaling are sufficient to cause cellular contraction. CHO-PLA1 cells transiently transfected with hPlexinD1 were allowed to spread on fibrinogen coated chamber slides and stained for imaging. Panel A shows a CHO-PD1 cell that expressed PlexinD1 at the membrane but was not exposed to hSema3E. Panel B shows a cell from the same cell line after exposure to hSema3E. As can be seen in Panel B, treatment with Sema3E causes cell collapse and possible endocytosis of the receptor.

FIG. 7 provides a graphed representation of experimental results demonstrating that Sema3E inhibits platelet adhesion to fibrinogen.

FIG. 8 provides images showing that Sema3E contracts pre-spread platelets. Human platelets were placed on chamber slides coated with fibrinogen and allowed to spread in the presence of 0.05 U/ml thrombin (spread platelets shown in Panel A). The platelets were then treated with a vehicle or media containing hSema3E (Panel B shows platelets post treatment with Sema3E). As can be appreciated by comparison of panel A and panel B, treating pre-spread platelets with hSema3E reversed cellular spreading, resulting in platelet contraction.

FIG. 9 and FIG. 10 provide two different graphed depictions of experimental results establishing that Sema3E inhibits α-Granule release from platelets.

FIG. 11 provides a graphed depiction of experimental results establishing that Sema3E (hSema3E) inhibits thrombin-induced P-Selectin translocation in human platelets.

FIG. 12 provides a graphed depiction of experimental results establishing that Sema3E (recombinant murine semaphorin 3E/Fc) inhibits induced P-Selectin translocation in mouse platelets induced by multiple agonists.

FIG. 13 depicts the results of a platelet aggregation study, showing that Sema3E inhibits platelet aggregation.

FIG. 14 provides a graphed depiction of experimental results establishing that Sema3E can both inhibit and reverse activation of α_{IIb}β₃ integrin.

FIG. 15 and FIG. 16 provide two different graphed depictions of experimental results establishing that Sema3E inhibits activation α_{IIb}β₃ integrin in mouse platelets induced by multiple agonists.

FIG. 17 and FIG. 18. provide two different graphed depictions of experimental results establishing that Sema3E inhibits clot formation in-vivo. FIG. 17 provides a graphical depiction of relative carotid artery flow observed in a mouse model of FeCl₃-induced carotid artery thrombosis, with the different lines representing relative carotid artery flow in mice receiving saline, mice receiving varying doses of heparin, and mice receiving Sema3E. FIG. 18 provides a graphic representation of the time to occlusion of the carotid artery observed in the same mouse model of FeCl₃-induced carotid artery thrombosis, with the different bars representing time to occlusion in mice receiving saline, mice receiving varying doses of heparin, and mice receiving Sema3E.

FIG. 19 provides representative images of ultrasound data obtained in the course of evaluating Sema3E in a mouse model of FeCl₃-induced carotid artery thrombosis

FIG. 20 provides experimental results showing that Sema3E inhibits activation of Rap1b (i.e., formation of GTP-Rap1b).

FIG. 21 provides the sequence of a recombinant human semaphorin 3E with CD33 Signal peptide and His tag (SEQ. ID. NO. 1).

FIG. 22 provides the sequence of a recombinant murine semaphorin 3E/Fc with CD33 signal peptide, linker and Fc (SEQ. ID. NO. 2).

FIG. 23 provides the sequence of a recombinant human semaphorin 3A/Fc with CD33 signal peptide, His tag, linker and Fc (SEQ. ID. NO. 3).

FIG. 24 provides the sequence of a recombinant murine semaphorin 3F/Fc with CD33 signal peptide, linker and Fc (SEQ. ID. NO. 4).

FIG. 25 provides the sequence of a recombinant human semaphorin 3E referred to as pCI-His8-hSema3E (SEQ. ID. NO. 5).

FIG. 26 provides the sequence of a recombinant human semaphorin 3E referred to as pCI-His8-hSema3E-convertase dead KARFAR (SEQ. ID. NO. 6).

### DETAILED DESCRIPTION OF THE INVENTION

Disclosed are materials, compositions, and components that can be used for, can be used in conjunction with, can be used in preparation for, or are products of the disclosed active agents, compositions and methods. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F, and an example of a combination molecule, A-D, is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the included claims.

It is also to be understood that the terminology used herein is for the purpose of describing particular embodiments only, and is not intended to limit the scope of the present invention which will be limited only by the appended claims.

Unless defined otherwise, all technical and scientific terms used herein have the meanings that would be commonly understood by one of skill in the art in the context of the present specification.

It must be noted that as used herein and in the appended claims, the singular forms “a,” “an,” and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. The term “patient” refers to a subject afflicted with a pathologic condition. The term “patient” includes human and veterinary subjects.

“Inhibit,” “inhibiting,” and “inhibition” mean to prevent, decrease, or inactivate an activity, response, condition, disease, or other biological parameter. This can include, but is not limited to the complete ablation of the activity, response, condition, or disease. This may also include, for example, a slowing or reduction of an activity, response, condition, disease, or other biological parameter as compared to a native level, with the term “native level” referring to a level evident in the absence of an inhibiting agent. In this context, a reduction can be any measurable reduction. In particular embodiments, a reduction can be, for example, a 10%, 20%, 30%, 40%, 50%, 60%, 70%, 80%, 90%, 100%, or any amount of reduction in between the specifically recited percentages, as compared to a native level.

As it is used herein in association with platelet activity, the term “reverse” specifically refers to reverting, at least in part, an activity, response, condition, disease, or other biological parameter associated with the presence or production of an agonist, such as thrombin, ADP, or thromboxane, such as thromboxane A2, to a state that more closely resembles the absence of the agonist. In this context, “reverting” can be a reduction in the activity, response, condition, disease, or other biological parameter associated with the presence or production of an agonist. In this context, a reduction can be any measurable reduction. In particular embodiments, a reduction can be, for example, a 10%, 20%, 30%, 40%, 50%, 60%, 70%, 80%, 90%, 100%, or any amount of reduction in between the specifically recited percentages, as compared to a native level. Therefore, the term “reverting” encompasses a complete reversal of an activity, response, condition, disease, or other biological parameter associated with the presence or production of an agonist.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be a 10%, 20%, 30%, 40%, 50%, 60%, 70%, 80%, 90%, 100%, or more, including any amount of increase in between the specifically recited percentages, as compared to native or control levels.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be used in providing a pharmaceutical formulation and can be selected to minimize any degradation of the active agent and to minimize any adverse side effects in the subject.

When referring to an active agent that is “biologically active” at a plexin receptor, the term “biologically active” refers to active agents which exhibit an association with a plexin receptor, such as active agents that bind to a plexin receptor, and wherein such association is coupled with inhibition or reversal of one or more platelet function, including one or more functions selected from platelet spreading, adhesion, aggregation, α-granule release, and clotting.

As used herein, the terms “treat,” “treating,” and “treatment” refer to a therapeutic benefit, whereby the detrimental effect(s) or progress of a particular pathologic condition, disease, condition, event or injury is prevented, reduced, halted, reversed or slowed.

A “therapeutically effective amount” is the amount of compound which achieves a therapeutic benefit, such as, for example, by inhibiting or reversing an activity, response, condition, disease, or other parameter associated with a pathologic condition. A therapeutically effective amount may be an amount which relieves, at least to some extent, one or more symptoms of a pathologic condition in a subject; returns to normal, either partially or completely, one or more physiological or biochemical parameters associated with or causative of a pathologic condition; and/or reduces the likelihood of the onset of a pathologic condition.

The terms “pathologic” or “pathologic conditions” refer to any deviation from a healthy, normal, or efficient condition which may be the result of a disease, condition, event or injury.

### I. Active Agents & Compositions

Active agents for inhibiting or reversing the activity of platelets are provided herein. In one embodiment, the active agents inhibit or reverse one or more platelet function. For example, in specific embodiments, the active agents describe herein inhibit or reverse one or more of platelet spreading, adhesion, aggregation, α-granule release, or clotting. In one embodiment, the active agent inhibits or reverses platelet spreading, adhesion, aggregation, α-granule release, and clotting. The active agents described herein may be a polypeptide agent. Specific examples of such polypeptide active agents include polypeptides belonging to or derived from semaphorin 3E proteins (“Sema3E polypeptides” or “Sema3E”). When discussing Sema3E polypeptides as contemplated herein, full-length Sema3E proteins, as well as derivatives, analogs and homologs of full-length Sema3E proteins are contemplated, provided that such polypeptides inhibit or reverse one, or more, or all of platelet spreading, adhesion, aggregation, α-granule release, or clotting. A “derivative” polypeptide molecule refers to a polypeptide formed from native compounds either directly or by modification or partial substitution. A “homolog” polypeptide molecule refers to a polypeptide product of a particular gene derived from a different species. An “analog” polypeptide molecule is a polypeptide that is similar in structure, but not identical, and differs with respect to number or nature of amino acids included in a referenced polypeptide sequence. For example, an analog to a given polypeptide will exhibit a level of sequence homology, but may include one or more amino acid substitutions or deletions.

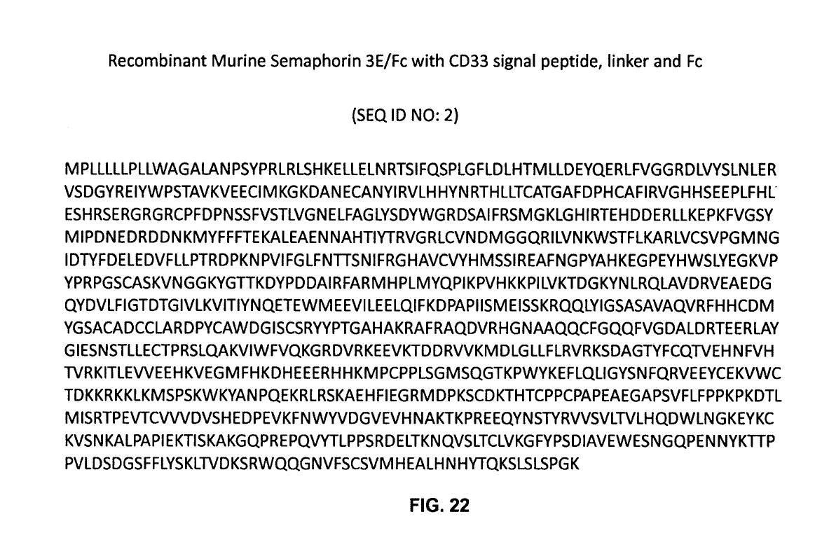

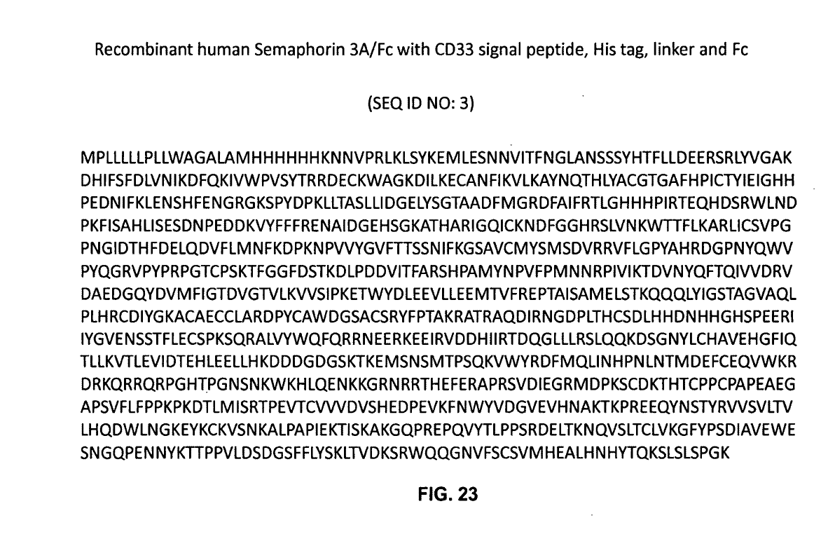

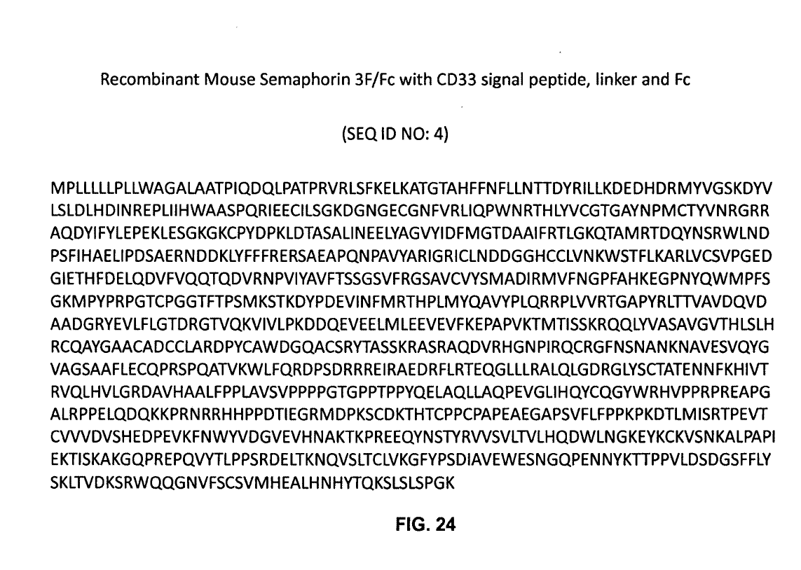

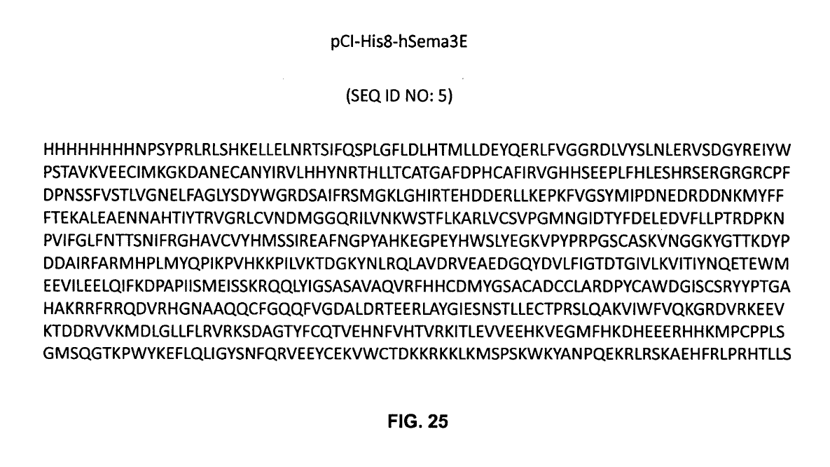

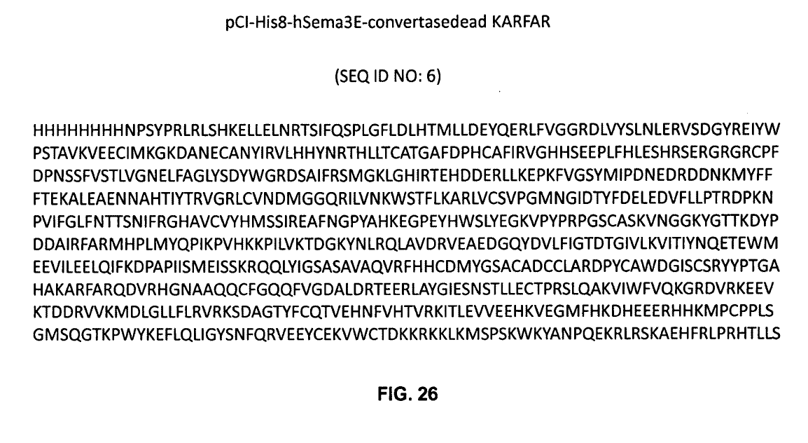

In specific embodiments, where the active agent is a Sema3E polypeptide, the active agent may be selected from mammalian Sema3E polypeptides, such as a mouse or human Sema3E polypeptide. Where the active agent is a mammalian Sema3E, the active agent may be selected from known, naturally occurring mammalian Sema3E polypeptides that have been isolated and purified according to techniques known in the art. Alternatively, a mammalian Sema3E as contemplated herein may be obtained through recombinant or synthetic production techniques well know in the art. Even further, the active agent may be selected from derivatives, analogs, or homologs of naturally occurring, recombinant, or synthetic mammalian Sema3E polypeptides. For example, in specific embodiments, the active agent may be selected from the recombinant human Sema3E detailed in FIG. 22 (“hSema3E”) (SEQ. ID. NO. 1) and the recombinant murine Sema 3E/Fc detailed in FIG. 23 (SEQ. ID. NO. 2), as well as derivatives, analogs and homologs of such polypeptides exhibiting the ability to inhibit or reverse one or more of platelet spreading, adhesion, aggregation, α-granule release, or clotting. In other embodiments, the active agent may be selected from the recombinant human Sema3E polypeptides detailed in FIG. 25 (SEQ. ID. NO. 5) and FIG. 26 (SEQ. ID. NO. 6), as well as derivatives, analogs and homologs of such polypeptides exhibiting the ability to inhibit or reverse one or more of platelet spreading, adhesion, aggregation, α-granule release, or clotting. In one embodiment, the active agent is selected from a derivative, homolog or analog of a naturally occurring mammalian Sema3E polypeptide, such as a full-length, naturally occurring human or mouse Sema3E, or one of the Sema polypeptides described by SEQ. ID. NO. 1, SEQ. ID. NO. 2, SEQ. ID. NO. 5, and SEQ. ID. NO. 6, with such active agent exhibiting a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring human or mouse Sema3E, or to SEQ. ID. NO. 1, SEQ. ID. NO. 2, SEQ. ID. NO. 5, or SEQ. ID. NO. 6. In another embodiment, the active agent is a derivative, homolog, or analog of full-length, naturally occurring human or mouse Sema3E or one of Sema 3E polypeptides detailed in SEQ. ID. NO. 1, SEQ. ID. NO. 2, SEQ. ID. NO. 5, and SEQ. ID. NO. 6, yet exhibits less polypeptide sequence homology to the polypeptide from which it is derived, such as, for example, a homology selected from one of 80% or less, 70% or less, 60% or less, or 50% or less, while retaining the ability to inhibit or reverse one or more of platelet spreading, adhesion, aggregation, α-granule release or clotting.

Semaphorins are known to interact with plexins, and plexin receptors are understood to be the predominant class of semaphorin receptors. There are four classes of plexins, class A-D, and it has been reported that class 3 semaphorins (e.g., Sema 3A, 3C, 3E, and 3F) interact with class A and class D plexins: Sema3A, Sema3C, and Sema3E are known to bind Plexin-D1; Sema3A and Sema3F are known to bind Plexin-A1, Plexin-A3, and Plexin-A4; and Sema3A is known to bind Plexin-A2. (Kruger R. et al. Semaphorins command cells to move. Nature Reviews. Volume 6. Pp 789-800. October 2005). It has been proposed that interactions between class 3 semaphorins and Plexin-A and Plexin-D are essential to the proper formation of vasculature, and it has been reported that class 3 semaphorin signaling mediated by Plexin-D1 is required for normal heart development. (Kruger R. et al. Semaphorins command cells to move. Nature Reviews. Volume 6. Pp 789-800. October 2005).

As is illustrated by the experimental examples provided herein, Plexin-D1 is expressed at the membrane of platelets, and binding of Sema3E to the Plexin-D1 receptor is sufficient to cause cellular contraction of cells allowed to spread on a fibrinogen containing matrix. Without being bound by a particular theory, therefore, it is believed that platelet function, including platelet spreading, adhesion, aggregation, α-granule release, and clotting, may be inhibited or reversed by activation of a plexin receptor, such as the Plexin-D1 receptor. Therefore, in particular embodiments, the active agents described herein are capable of binding to and are biologically active at one or more plexin receptors. In one such embodiment, the active agent is a Sema3E polypeptide as described herein that binds to and is biologically active at one or more plexin receptors selected from Plexin-D1 and the Plexin-A class of receptors (i.e., Plexin-A1 through Plexin-A4). In one such specific embodiment, the active agent is selected from a Sema3E polypeptide as described herein that binds to and is biologically active at a Plexin-D1 receptor.

Sema3E polypeptides that are capable of binding to and are biologically active at one or more plexin receptors, as described herein, can be selected from mammalian Sema3E polypeptides, such as a mouse or human Sema3E. Where the active agent is a mammalian Sema3E, the active agent may be selected from naturally occurring mammalian Sema3E polypeptides that have been isolated and purified from their natural environment. Alternatively, a mammalian Sema3E as contemplated herein may be obtained through recombinant or synthetic production techniques well know in the art. Even further, the active agent may be selected from derivatives, analogs, or homologs of naturally occurring, recombinant, or synthetic mammalian Sema3E polypeptides. For example, in specific embodiments, a Sema3E active agent capable of binding to and biologically active at a plexin receptor as described herein may be selected from the recombinant Sema3E polypeptides provided in SEQ. ID. NO. 1, SEQ. ID. NO. 2, SEQ. ID. NO. 5, and SEQ. ID. NO. 6, as well as derivatives, analogs and homologs of such polypeptides that bind to and are biologically active at the desired plexin receptor(s). In one such embodiment, the active agent is selected from a derivative, homolog or analog of a naturally occurring mammalian Sema3E polypeptide, such as a full-length, naturally occurring human or mouse Sema3E, or one of the Sema polypeptides described by SEQ. ID. NO. 1, SEQ. ID. NO. 2, SEQ. ID. NO. 5, and SEQ. ID. NO. 6, with such active agent exhibiting a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring human or mouse Sema3E, or to SEQ. ID. NO. 1, SEQ. ID. NO. 2, SEQ. ID. NO. 5, or SEQ. ID. NO. 6. In another embodiment, the active agent is a derivative, homolog, or analog of full-length, naturally occurring human or mouse Sema3E or one of SEQ. ID. NO. 1, SEQ. ID. NO. 2, SEQ. ID. NO. 5, and SEQ. ID. NO. 6, yet exhibits less polypeptide sequence homology to the polypeptide from which it is derived, such as, for example, a homology selected from one of 80% or less, 70% or less, 60% or less, or 50% or less, while retaining the ability to bind to and exhibit biological activity at the desired plexin receptor(s).

Rap1b activation is a common feature of platelet activation via various different agonists. (See, e.g., Wei, A. et al. New Insights into the haemostatic function of platelets. Br J. Heamatol. 2009 Jul. 28; Chrzanowska-Wodnicka, M. et al. Rap1b is required for normal platelet function and hemostasis in mice. J. Clin. Invest. 115: 680-687 (2005)). A critical step in platelet activation regulated by Rap1b is α_{IIb}β₃ integrin activation, and Rap1b deficiency impairs soluble fibrinogen binding induced by multiple agonists, indicating that Rap1b is involved upstream from α_{IIb}β₃ integrin activation. Rap1b function is required for the normal α_{IIb}β₃ integrin signaling, and a loss of Rap1b function has been associated with protection against thrombosis. (See, e.g., Wei, A. et al. New Insights into the haemostatic function of platelets. Br J Heamatol. 2009 Jul. 28; Chrzanowska-Wodnicka, M. et al. Rap1b is required for normal platelet function and hemostasis in mice. J. Clin. Invest. 115: 680-687 (2005)). As is highlighted in the experimental examples provided herein, embodiments of active agents described herein are capable of inhibiting platelet function mediated by several agonists, including thrombin, ADP, and thromboxane, such as thromboxane A2. Moreover, active agents as described herein inhibit platelet spreading and adhesion on β₁ and β₃ substrates.

Without being bound by a particular theory, it is believed that the actions of the active agents described herein are not observed to be agonist dependent because, in specific embodiments, the active agents according to the present description inhibit activation of Rap1b. As used in the context of the present disclosure, “activation of Rap1b” refers to formation of GTP-Rap1b. Again, α_{IIb}β₃ integrin activation is important to platelet activation and function, and Rap1b deficiency, such as may occur by inhibiting activation of Rap1b impairs soluble fibrinogen binding induced by multiple agonists. Therefore, in particular embodiments, the active agents described herein may be selected from Sema3E capable of inhibiting activation of Rap1b, resulting in inhibition of α_{IIb}β₃ integrin activation.

Sema3E polypeptides that are capable of inhibiting activation of Rap1b, can be selected from mammalian Sema3E polypeptides, such as a mouse or human Sema3E. Where the active agent capable of inhibiting activation of Rap1b is a mammalian Sema3E, the active agent may be selected from naturally occurring mammalian Sema3E polypeptides that have been isolated and purified from their natural environment. Alternatively, a mammalian Sema3E as contemplated herein may be obtained through recombinant or synthetic production techniques well know in the art. Even further, a Sema3E active agent capable of inhibiting activation of Rap1b may be selected from derivatives, analogs, or homologs of naturally occurring, recombinant, or synthetic mammalian Sema3E polypeptides. For example, in specific embodiments, a Sema3E active agent capable of inhibiting activation of Rap1b may be selected from the recombinant Sema3E polypeptides detailed in SEQ. ID. NO. 1, SEQ. ID. NO. 2, SEQ. ID. NO. 5, and SEQ. ID. NO. 6, as well as derivatives, analogs and homologs of such polypeptides that inhibit activation of Rap1b. In one such embodiment, the active agent is selected from a derivative, homolog or analog of a naturally occurring mammalian Sema3E polypeptide, such as a full-length, naturally occurring human or mouse Sema3E, or one of the Sema polypeptides described by SEQ. ID. NO. 1, SEQ. ID. NO. 2, SEQ. ID. NO. 5, and SEQ. ID. NO. 6, with such active agent exhibiting a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring human or mouse Sema3E, or to SEQ. ID. NO. 1, SEQ. ID. NO. 2, SEQ. ID. NO. 5, and SEQ. ID. NO. 6. In another embodiment, the active agent is a derivative, homolog, or analog of full-length, naturally occurring human or mouse Sema3E or one of SEQ. ID. NO. 1, SEQ. ID. NO. 2, SEQ. ID. NO. 5, and SEQ. ID. NO. 6, yet exhibits less polypeptide sequence homology to the polypeptide from which it is derived, such as, for example, a homology selected from one of 80% or less, 70% or less, 60% or less, or 50% or less, while retaining the ability to inhibit activation of Rap1b.

A polypeptide of a desired structure can be produced using methods and materials well known in the art. For example, various methods for isolating naturally occurring polypeptides or producing recombinant polypeptides are well known. Moreover, various methods are known for synthetically producing a polypeptide of desired sequence. For example, peptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide corresponding to a desired protein can be synthesized by standard chemical reactions. For example, a peptide can be synthesized and not cleaved from its synthesis resin whereas another peptide fragment of a protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, a desired protein or peptide can be synthesized in-vivo using standard recombinant techniques. Where independent peptides that are to be linked to form a desired protein are independently produced in-vivo, once such independent peptides are produced and isolated, they may be linked to form a desired protein or fragment thereof via similar peptide condensation reactions.

For example, enzymatic ligation of cloned or synthetic peptide segments allow relatively short peptide fragments to be joined to produce larger peptide fragments, polypeptides or whole protein domains. (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction. (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Compositions including an active agent as described herein are also provided. Such compositions may include one or more active agents as described herein. In one embodiment, a composition is prepared as a pharmaceutical formulation. For example, in addition to one or more active agent as described herein, a pharmaceutical formulation may include a pharmaceutically acceptable carrier and/or one or more pharmaceutically acceptable excipients to provide a formulation that is suitable for therapeutic administration. As used herein, “pharmaceutically acceptable” refers to a material that is not biologically or otherwise undesirable, e.g., the material is suitable for administration to a subject together with the desired active agent (e.g., a desired active agent as described herein) and is compatible with other components of the pharmaceutical formulation in which it is contained. The carrier and any excipient(s) would naturally be selected to minimize any degradation of the active agent or adverse side effects in the subject.

A pharmaceutical formulation according to the present description may be prepared in any form suitable for administration, such as a tableted composition, a powder composition for encapsulation, a solution composition for encapsulation or parenteral delivery, an emulsion, or a suspension, such as a formulation that incorporates or is incorporated into, for example, microparticles, a matrix material, or liposomes. A pharmaceutical formulation as described herein may include components targeted to a particular cell type via antibodies, receptors, or receptor ligands. Pharmaceutical carriers and excipients and their formulations are well described in the literature, including, for example, in Remington: The Science and Practice of Pharmacy (19th ed.) ed. A. R. Gennaro, Mack Publishing Company, Easton, Pa. 1995.

Where appropriate, a pharmaceutically-acceptable salt may be used in the pharmaceutical formulation to render the formulation isotonic. Examples of liquid pharmaceutically-acceptable carriers include, but are not limited to, saline, Ringer's solution, and dextrose solution. Where the pharmaceutical formulation is provided as a solution or suspension, particularly for parenteral delivery, the pH of the formulation can be adjusted as desired to facilitate delivery to a subject and/or preservation of the active agent or other formulation components. Carriers and excipients suitable for preparing pharmaceutical formulations include, for example, a well-known variety of pharmaceutically acceptable polymers, saccharides, salts, lipids, phospholipids, surfactants, gels, polypeptides, and amino acids. The pharmaceutical formulation according to the present description may include sustained release preparations. It will be apparent to those persons skilled in the art that certain carriers and/or excipients may be more preferable depending upon, for instance, the route of administration and concentration of composition being administered. A pharmaceutical formulation as described herein may include one or more thickener, flavoring, diluent, buffer, preservative, antimicrobial agents, antiinflammatory agents, anesthetics, surface active agent, and the like.

The herein disclosed compositions, including pharmaceutical formulations, may be administered in a number of ways depending on whether local or systemic treatment is desired, and on the area to be treated. Parenteral administration of the composition, if used, is generally characterized by injection. Injectables can be prepared in conventional forms, either as liquid solutions or suspensions, solid forms suitable for dissolution or suspension in liquid prior to injection, or as emulsions. A revised approach for parenteral administration involves use of a slow release or sustained release system such that a constant dosage is maintained. (See, e.g., U.S. Pat. No. 3,610,795, which is incorporated by reference herein.)

The exact amount of a given composition required to achieve a therapeutic affect will vary from subject to subject, depending on the species, age, weight and general condition of the subject, the severity of the pathologic condition being treated, the particular active agent used, its mode of administration, and the like. The dosage ranges for the administration of the compositions are those large enough to produce a therapeutic effect. The dosage can be adjusted to avoid or reduce the occurrence of adverse side effects, such as unwanted cross-reactions, anaphylactic reactions, and the like. The dosage may vary with the age, condition, sex and extent of the disease in the patient, route of administration, or whether other drugs are included in the regimen. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

### II. Methods of Modulating Platelet Function, Treatment & Screening

Methods for inhibiting platelet function are provided herein. In one embodiment, a method for inhibiting platelet function includes treating one or more platelets with an active agent as described herein. In one such embodiment, the step of treating one or more platelets may be carried out by administering to a patient in need thereof a therapeutically effective amount of an active agent as described herein. Where desired, the active agent may be administered using a composition as described herein. In particular embodiments, treatment of the one or more platelets with the active agent inhibits or reverses one or more of platelet spreading, adhesion, aggregation, α-granule release, and clotting. In one such embodiment, treatment of the one or more platelets with the active agent inhibits each of platelet spreading, adhesion, aggregation, α-granule release, and clotting. The active agent may be selected from the active agents described herein and administration of the active agent may be accomplished by administration of such active agent using a composition as described herein. In a specific embodiment, a method for inhibiting platelet function includes administering an active agent as described herein, wherein administering the active agent inhibits hemostasis and thrombosis associated with platelet function.

In another embodiment, the methods of the present invention include treating a patient at risk for or suffering from a pathologic condition associated with one or more platelet functions, wherein the method includes administering to such patient a therapeutically effective amount of an active agent as described herein. Pathologic conditions that are associated with platelet function or activity may be selected from pathologic conditions associated with one or more of platelet spreading, adhesion, aggregation, α-granule release or clotting, including, by way of example and not limitation, one or more of stroke, myocardial infarction, unstable angina, angina, thrombosis, including deep venous thrombosis, pulmonary embolism, and atrial fibrillation. Therefore, in particular embodiments, the methods of the present invention include identifying a patient at risk of or suffering from one or more of stroke, myocardial infarction, unstable angina, angina, thrombosis, including deep venous thrombosis, pulmonary embolism, and atrial fibrillation, and administering to the patient a therapeutically effective amount of an active agent as described herein capable of inhibiting platelet activity. In specific embodiments, the active agent inhibits one or more platelet function selected from platelet spreading, adhesion, aggregation, α-granule release, and clotting. In another embodiment of the methods of treatment described herein, administering a therapeutically effective amount of an active agent as described herein inhibits each of platelet spreading, adhesion, aggregation, α-granule release, and clotting. In each of the embodiments of the methods associated with inhibition of platelet function, the active agent may be selected from the active agents described herein, and administering a therapeutically effective amount of the active agent may be accomplished by administration of such active agent using a composition as described herein.

In yet another embodiment, the methods of the present invention include reversing platelet activity. In such an embodiment, platelets exhibiting one or more of platelet spreading, aggregation, adhesion, or clotting are treated with an active agent as described herein. The active agent may be selected from the active agents described herein, and treatment with the active agent may be accomplished by administration of such active agent using a composition as described herein. In particular embodiments, treatment with the active agent results in reversal of one or more of platelet spreading, aggregation, adhesion, α-granule release or clotting, and in one embodiment, treatment with the active agent results in reversal of each of platelet spreading, aggregation, adhesion, and clotting. In further embodiments, such methods may include treating a patient suffering from a pathologic condition associated with one or more platelet functions, wherein the method includes administering to such patient a therapeutically effective amount of an active agent as described herein. Therefore, in particular embodiments, the methods of the present invention include identifying a patient suffering from a pathologic condition associated with one or more of platelet spreading, adhesion, aggregation, α-granule release or clotting, including, by way of example and not limitation, one or more of stroke, myocardial infarction, unstable angina, angina, thrombosis, including deep venous thrombosis, pulmonary embolism, and atrial fibrillation, and administering to the patient a therapeutically effective amount of an active agent as described herein capable of reversing platelet activity, wherein administering the active agent results in a reversal of one, or more, or each of platelet spreading, adhesion, aggregation, α-granule release or clotting.

Also described herein, are methods of treating a patient at risk for or suffering from a pathologic condition associated with platelet function, wherein the method involves inhibiting activation of Rap1b. As has already been described, Rap1b activation is a common feature of platelet activity, and is believed to be necessary for activation of α_{IIb}β₃ integrin. In one such embodiment, therefore, a method for treating a subject at risk for or suffering from a pathologic condition associated with platelet function includes inhibiting activation of Rap1b, wherein the step of inhibiting activation of Rap1b includes administering an active agent as described herein. For example, in particular embodiments, a method for treating a subject at risk for or suffering from a pathologic condition associated with platelet function is provided, wherein the method includes administering a therapeutically effective amount of an active agent selected from a Sema3E polypeptide as described herein to a subject in need thereof. The Sema3E polypeptide can be formulated and administered to a subject using a composition as described herein. The pathologic condition treated by a method involving inhibition of activation of Rap1b can be selected from conditions resulting from or associated with one or more of platelet spreading, adhesion, aggregation, α-granule release or clotting, including, by way of example and not limitation, stroke, myocardial infarction, unstable angina, angina, thrombosis, including deep venous thrombosis, pulmonary embolism, and atrial fibrillation.

In yet another embodiment, a method of treating a subject at risk for or suffering from a pathologic condition associated with platelet function includes administering an active agent that binds to and is biologically active at one or more plexin receptors. In one such embodiment, therefore, a method for treating a patient at risk for or suffering from a pathologic condition associated with platelet function includes administering an active agent that binds to and is biologically active at one or more plexin receptors. In specific embodiments of such methods, the active agent may be selected from the Sema3E active agents described herein and the plexin receptor may be selected from, for example, one or more of Plexin-D1 or a Plexin-A receptor, such as one or more of Plexin-A1 through Plexin-A4. In such embodiments, the Sema3E active agent may be formulated and administered to a subject using a composition as described herein. In yet another specific embodiment, a method for treating a patient at risk for or suffering from a pathologic condition associated with platelet function includes administering an active agent that binds to and is biologically active at Plexin D1, wherein the active agent is a Sema3F polypeptide as described herein. The pathologic condition treated by a method involving administration of an active agent that is biologically active at one or more plexin receptors can be selected from conditions resulting from or associated with one or more of platelet spreading, adhesion, aggregation, α-granule release or clotting, including, by way of example and not limitation, stroke, myocardial infarction, unstable angina, angina, thrombosis, including deep venous thrombosis, pulmonary embolism, and atrial fibrillation.

Methods of screening for or evaluating an agent that inhibits or reverses platelet function are provided herein. For example, in particular embodiments, methods of screening for active agents according to the present description can be carried out using the in-vitro experiments and in-vivo models described herein. In an alternative embodiment, a method for screening an active agent capable of inhibiting or reversing platelet function includes determining the ability of said agent to bind one or more plexin receptor(s) and exhibit biological activity at such receptor. In such a method, the plexin receptor may be selected from, for example, Plexin-D1 and the Plexin-A family of receptors. In a specific embodiment, a screening method may include the following steps: contacting a first cell expressing one or more plexin receptors with a candidate agent, contacting a second cell essentially identical to the first cell but substantially lacking a plexin receptor with the candidate agent, and assaying for binding to or biological activity of the plexin receptor in the first and second cells, wherein detectably higher plexin binding and activity in the first cell as compared to the second cell indicates activation by said agent. In carrying out such a screening method, the cells used may be, for example CHO cells, and the plexin expressing cells may be CHO cells transiently transfected with a targeted plexin receptor. Detection of plexin binding and activation can be carried out by one or more of a combination if immunocytological techniques and cellular morphology analytical methods.

In yet a further embodiment of a method of screening active agents as described herein, the method may include evaluating the ability of an active agent to inhibit activation of Rap1b. Such a method may include steps such as those exemplified in Example 13. For example, the method may include providing a proposed active agent, providing a commercially available kit for assaying activation of Rap1b, and assaying the inhibition of Rap1b activation in the presence of the proposed active agent.

### III Examples

The Examples that follow are offered for illustrative purposes only and are not intended to limit the scope of the compositions and methods described herein in any way. It is to be understood that the disclosed compositions and methods are not limited to the particular methodologies, protocols, and reagents described herein. In each instance, unless otherwise specified, standard materials and methods were used in carrying out the work described in the Examples provided. All patent and literature references cited in the present specification are hereby incorporated by reference in their entirety.

The practice of the present invention employs, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, T., et al. (1982) Molecular Cloning: A Laboratory Manual (Cold Spring Harbor Laboratory, Cold Spring Harbor, N.Y.); Sambrook, J., et al. (1989) Molecular Cloning: A Laboratory Manual, 2^{nd} Ed. (Cold Spring Harbor Laboratory, Cold Spring Harbor, N.Y.); Ausubel, F. M., et al. (1992) Current Protocols in Molecular Biology, (J. Wiley and Sons, NY); Glover, D. (1985) DNA Cloning, I and II (Oxford Press); Anand, R. (1992) Techniques for the Analysis of Complex Genomes, (Academic Press); Guthrie, G. and Fink, G. R. (1991) Guide to Yeast Genetics and Molecular Biology (Academic Press); Harlow and Lane (1988) Antibodies: A Laboratory Manual (Cold Spring Harbor Laboratory, Cold Spring Harbor, N.Y.); Jakoby, W. B. and Pastan, I. H. (eds.) (1979) Cell Culture. Methods in Enzymology, Vol. 58 (Academic Press, Inc., Harcourt Brace Jovanovich (NY); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (eds) (1994) Manipulating the Mouse Embryo; A Laboratory Manual, 2^{nd} Edition, Cold Spring Harbor Laboratory Press, Cold Spring Harbor, N.Y.). A general discussion of techniques and materials for human gene mapping, including mapping of human chromosome 1, is provided, e.g., in White and Lalouel (1988) Ann. Rev. Genet. 22:259 279. The practice of the present invention employs, unless otherwise indicated, conventional techniques of chemistry, molecular biology, microbiology, recombinant DNA, genetics, and immunology. (See, e.g., Maniatis et al., 1982; Sambrook et al., 1989; Ausubel et al., 1992; Glover, 1985; Anand, 1992; Guthrie and Fink, 1991).

Nothing herein is to be construed as an admission that the subject matter taught herein is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### Example 1

### PlexinD1 is Expressed at the Membrane of Platelets

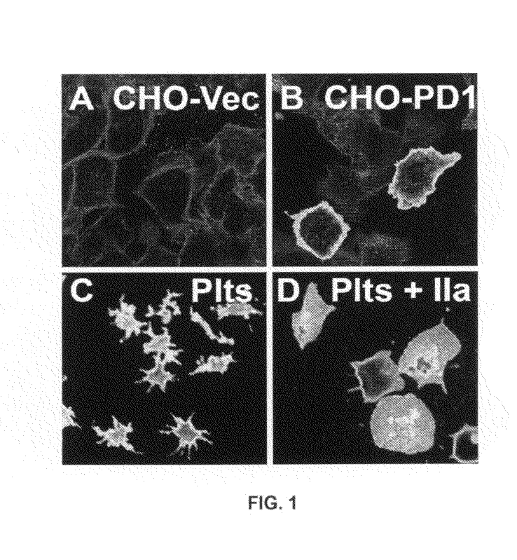

PlexinD1 is expressed at the cellular membrane of platelets. As a control, CHO-Vec cells and CHO-PLA1 cells transiently transfected with hPlexinD1 (CHO-PD1) were allowed to spread on fibrinogen coated chamber slides, and fixed cells were stained with alexa 544-Phaloidin (Molecular Probes, Eugene, Oreg.) and goat anti-plexinD1 (R&D systems, Minneapolis, Minn.) for 1 hour at room temperature, followed by donkey-anti-goat alexa 488-IgG (Molecular Probes, Eugene, Oreg.) for 1 hour at RT. Cells were imaged using confocal microscopy. As can be seen in panel B of FIG. 1, CHO-PD1 cells, which express the PlexinD1 receptor, exhibited anti-PlexinD1 staining at the cell membrane.

In separate experiments, human and mouse platelets were allowed to spread on fibrinogen coated chamber slides, and as was done with the CHO-Vec and CHO-PD1 cells, the fixed platelet cells were stained with alexa 544-Phaloidin (Molecular Probes, Eugene, Oreg.) and goat anti-plexinD1 (R&D systems, Minneapolis, Minn.) for 1 hour at RT, followed by donkey-anti-goat alexa 488-IgG (Molecular Probes, Eugene, Oreg.) for 1 hour at RT. Again, cells were imaged using confocal microscopy. Anti-PlexinD1 staining was observed at the cell membrane in human and mouse platelets, panels C and D of FIG. 1, showing human platelets exhibiting anti-PlexinD1 staining

### Example 2

### Semaphorin 3E is a Highly Potent Inhibitor of Platelet Spreading

8 well borosilicate chamber slides (Nalge Nunc International, Rochester, N.Y.) were coated with 100 ug/ml fibrinogen overnight at 4° C. followed by blocking with 0.5% human serum albumin for 30 minutes at 37° C. Chamber slides were washed 3× with HBSS. Human platelets (2×10⁷) in M199 were incubated with one of a vehicle or a media containing one of Netrin 1 (at 7.5 μg/ml, 5 μg/ml and 25 μg/ml concentrations), Netrin 4 (at 7.5 μg/ml, 5 μg/ml and 25 μg/ml concentrations), Slit 2 (at 8 μg/ml, 5 μg/ml, 24 μg/ml and 40 μg/ml concentrations), hSema3E (SEQ ID NO: 1) (at 1 μg/ml, 2 μg/ml, 5 μg/ml, 10 μg/ml, and 25 ug/ml), mouse Sema3F/Fc (SEQ ID NO: 4) (at 10 μg/ml and 25 μg/ml concentrations), hSema3A/Fc (SEQ ID NO: 3) (at 10 μg/ml and 25 μg/ml concentrations), for 10 minutes at room temperature, and then they were placed on the coated chamber slides for 30 minutes at 37° C., 5% CO₂ in the presence of 0.05 U/ml thrombin or 20 m/ml ADP. Platelets were fixed for 20 minutes with 2% PFA and washed three times with HBSS. Morphologic study of adhered platelets was performed by staining platelets with Alexa 488-phaloidin and 544-WGA followed by imaging using confocal microscopy. Adhesion assays were performed by counting platelets in 5 high powered fields.

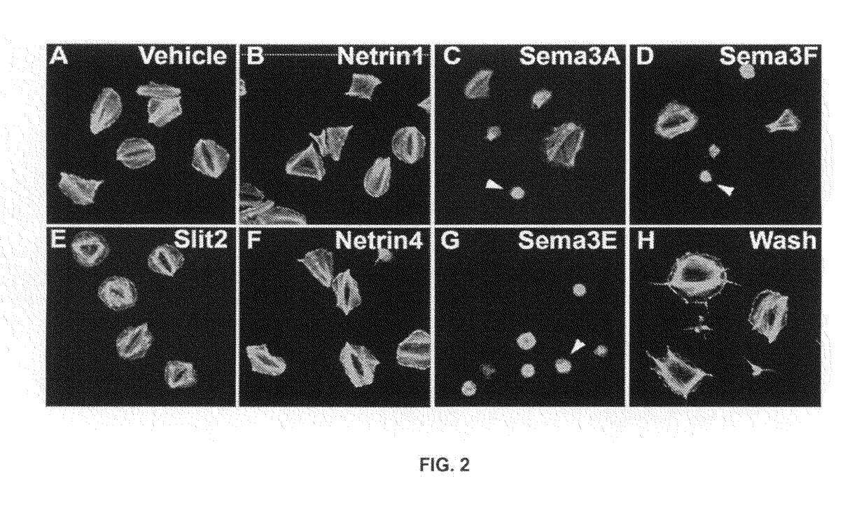

Exemplary images of the results obtained are provided in panels A-H of FIG. 2. As is illustrated in FIG. 2, no affect on platelet spreading was observed in platelets exposed to the various concentrations of Netrin 1, Netrin 4 or Slit 2. Platelets exposed to semaphorins exhibited significant reduction in spreading, with Sema3E (exemplified by results shown in panel G) providing a particularly potent effect. It was observed that Sema3E effectively inhibited platelet spreading at concentrations as low as 1 μg/ml and provided a dose dependent increase in inhibition as the concentration rose to 25 μg/ml. Sema3F inhibited platelet spreading starting at about 10 μg/ml, but provided increased inhibition of platelet spreading as the concentration rose to 25 μg/ml. Sema3A provided only a minimal effect on platelet spreading at 10 μg/ml but exhibited increased effect as the dose rose to 25 μg/ml.

In a further step, hSema3E treated platelets were seeded on fibrinogen coated chamber slides and allowed to spread for 10 minutes. Then hSema3E containing media was removed, and cells were washed once with M199. M199 containing 0.05 U/ml thrombin was then added to platelets. Platelets were then allowed to spread for 30 minutes. As can be seen in panel H of FIG. 2, the effects of hSema3E were reversible, as removal and washing of the hSema3E containing media restored the platelets ability to spread.

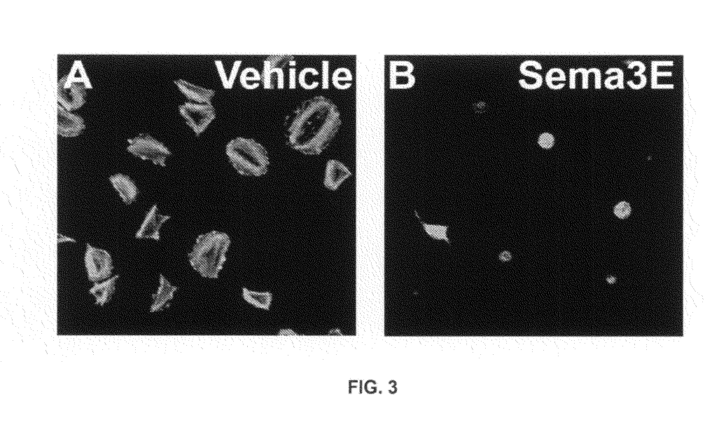

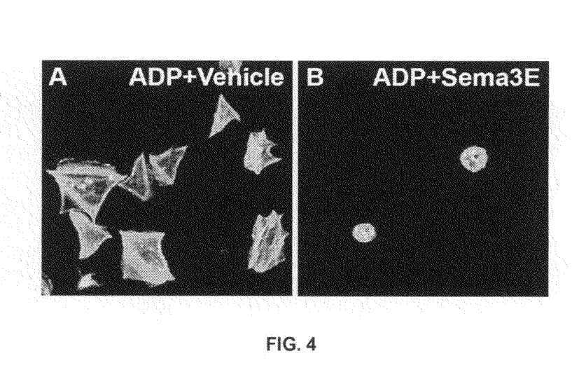

The effect of Sema3E was preserved in mouse platelets and was observed not to be agonist dependent. As is shown in FIG. 3, hSema3E inhibited spreading of mouse platelets when such platelets were treated under the same conditions as described above in association with human platelets. Moreover, as is illustrated by comparison of panels A and B of FIG. 4, Sema3E not only inhibited platelet spreading in the presence of thrombin, but hSema3E also inhibited ADP induced platelet spreading.

Two additional human Sema3E polypeptides (SEQ. ID. NO. 5 and SEQ. ID. NO. 6) were evaluated in for their ability to inhibit spreading of human and mouse platelets under similar conditions as those described above. Both such Sema3E polypeptides provided effects comparable to those achieved by hSema3E (SEQ. ID. NO. 1) reported herein.

### Example 3

### Sema3E Inhibits Attachment and Spreading on β₃ and β₁ Integrin Dependent Substrates

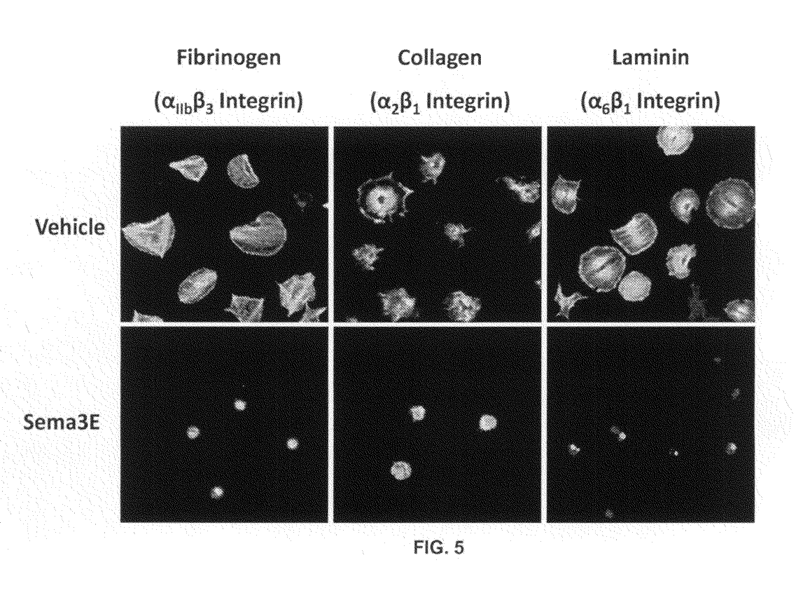

To evaluate the ability of Sema3E to inhibit attachment and spreading of platelets on different substrates, 8 well borosilicate chamber slides (Nalge Nunc International, Rochester, N.Y.) were coated with one of 100 ug/ml fibrinogen (α_{IIb}β₃ integrin), 100 ug/ml collagen (α₂β₁ integrin), and 100 ug/ml laminin (α₆β₁ integrin) overnight at 4° C. followed by blocking with 0.5% human serum albumin for 30 minutes at 37° C. Chamber slides were washed 3× with HBSS. Human platelets (2×10⁷) in M199 were incubated with vehicle or media containing hSema3E (SEQ ID NO: 1) at a concentration of 5μ, for 10 minutes at room temperature, and then they were placed on the coated chamber slides for 30 minutes at 37° C., 5% CO₂ in the presence of 0.05 U/ml thrombin. Platelets were fixed for 20 minutes with 2% PFA and washed three times with HBSS. Morphologic study of adhered platelets was performed by staining platelets with Alexa 488-phaloidin and 544-WGA followed by imaging using confocal microscopy. Adhesion assays were performed by counting platelets in 5 high powered fields. hSema3E worked to inhibit platelet spreading (shown in FIG. 5) and attachment on each of the substrates.

### Example 4

### Sema3E/PlexinD1 Signaling Sufficient to Cause Cellular Contraction

In order to confirm that activation of PlexinD1 by Sema3E was sufficient to inhibit cellular spreading, CHO-PLA1 cells were transiently transfected with hPlexinD1 and the effect of hSema3E (SEQ ID NO: 1) on such cells was evaluated. CHO-PLA1 cells transiently transfected with hPlexinD1 or Platelets were allowed to spread on fibrinogen coated chamber slides as previously mentioned, fixed cells were stained with alexa 544-Phaloidin (Molecular Probes, Eugene, Oreg.) and goat anti-plexinD1 (R&D systems, Minneapolis, Minn.) for 1 hour at RT followed by donkey-anti-goat alexa 488-IgG (Molecular Probes, Eugene, Oreg.) for 1 hour at RT. Cells were imaged using confocal microscopy.

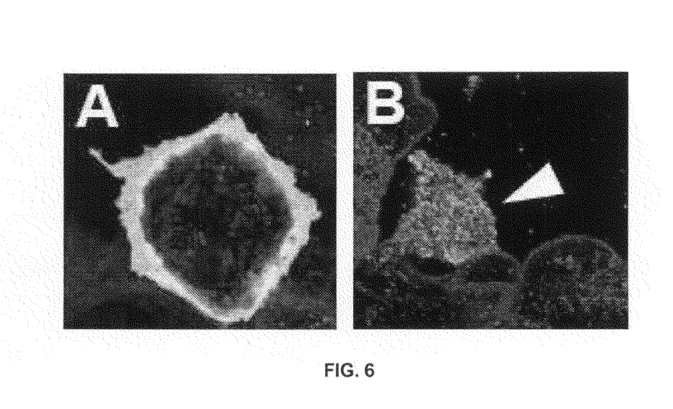

Such cells were then exposed to media containing hSema3E at a concentration of 5 μg/ml. Panel A of FIG. 5 shows a CHO-PD1 cell that expressed PlexinD1 at the membrane but was not exposed to hSema3E. Panel B of FIG. 6 shows a cell from the same cell line (CHO-PDI, expressing PlexinD1 at the membrane) after exposure to hSema3E. As can be seen in panel B, treatment with Sema3E causes cell collapse and possible endocytosis of the receptor.

### Example 5

### Sema3E Inhibits Platelet Adhesion to Fibrinogen

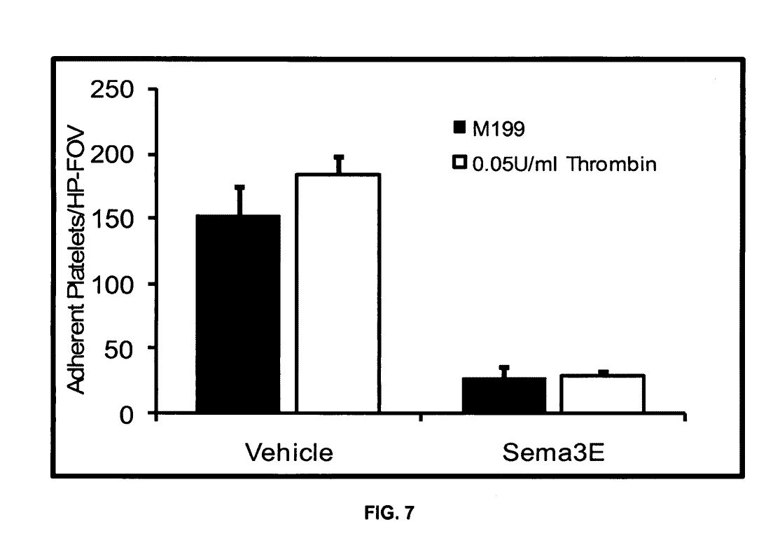

8 well borosilicate chamber slides (Nalge Nunc International, Rochester, N.Y.) were coated with 100 μg/ml fibrinogen, overnight at 4° C. followed by blocking with 0.5% human serum albumin for 30 minutes at 37° C. Chamber slides were washed 3× with HBSS. Platelets (2×10⁷) in M199 were incubated in the presence of a vehicle or media including 10 μg/ml hSema3E (SEQ ID NO: 1) for 10 minutes at room temperature, and then they were placed on the coated chamber slides for 30 minutes at 37° C., 5% CO₂ in the presence or absence of 0.05 U/ml thrombin. Platelets were fixed for 20 minutes with 2% PFA and washed three times with HBSS. Adhesion assays were then performed by counting platelets in 5 high powered fields. The results are provided in FIG. 7, which illustrates that treating the platelets with hSema3E inhibits platelet adhesion to fibrinogen.

Two additional human Sema3E polypeptides (SEQ. ID. NO. 5 and SEQ. ID. NO. 6) were evaluated in for their ability to inhibit adhesion of human platelets to fibrinogen under conditions similar to those described above. Both such Sema3E polypeptides provided effects comparable to those achieved by hSema3E (SEQ. ID. NO. 1) reported herein.

### Example 6

### Sema3E Contracts Pre-Spread Platelets

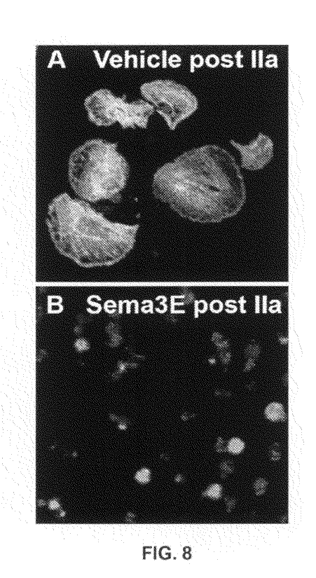

8 well borosilicate chamber slides (Nalge Nunc International, Rochester, N.Y.) were coated with 100 μg/ml fibrinogen overnight at 4° C. followed by blocking with 0.5% human serum albumin for 30 minutes at 37° C. Chamber slides were washed 3× with HBSS. Human platelets were placed on the coated chamber slides and allowed to spread for 10 minutes at 37° C., 5% CO₂ in the presence of 0.05 U/ml thrombin. Then the platelets were treated with a vehicle or media containing hSema3E (SEQ ID NO: 1) at a concentration of 10 μg/ml and allowed to spread for an additional 20 minutes at 37° C. Platelets were fixed for 20 minutes with 2% PFA and washed three times with HBSS. Morphologic study of adhered platelets was performed by staining platelets with Alexa 488-phaloidin and 544-WGA, followed by imaging using confocal microscopy. As is can be appreciated by comparison of panel A and panel B of FIG. 8, treating pre-spread platelets with hSema3E reversed cellular spreading, resulting in platelet contraction.

### Example 7

### Sema3E Inhibits α-Granule Release

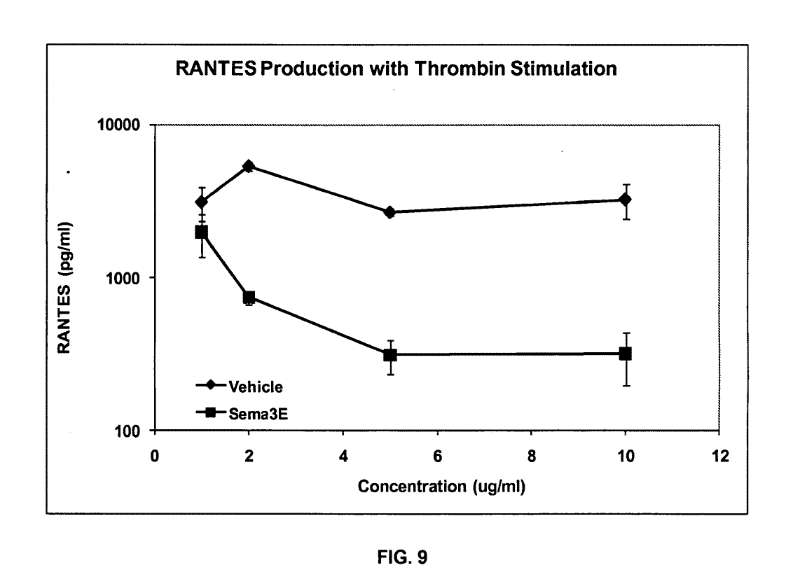

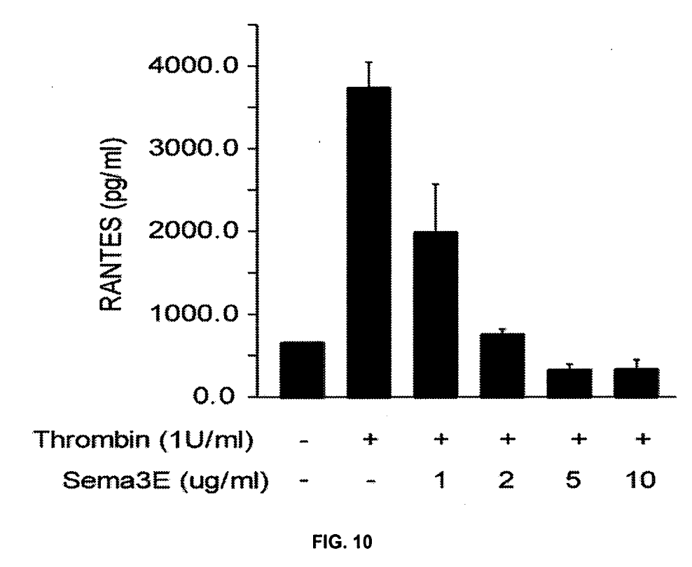

Washed human platelets were treated with either a vehicle or media containing varied concentrations of hSema3E (SEQ ID NO: 1) for 10 minutes at room temperature, followed by treatment with 0.05 U/ml thrombin for an additional 10 minutes at RT. Platelets were centrifuged at 500 g for 10 minutes, and supernatants were removed and used for RANTES ELISA following manufactures protocol (human Rantes DuoSet Elisa, R&D systems, Minneapolis, Minn.). The results of the RANTES ELISA are provided in FIG. 9 and FIG. 10, which illustrate that hSema3E significantly reduced RANTES production in the presence of thrombin.

### Example 8

### Sema3E Inhibits P-Selectin Translocation in Human Platelets

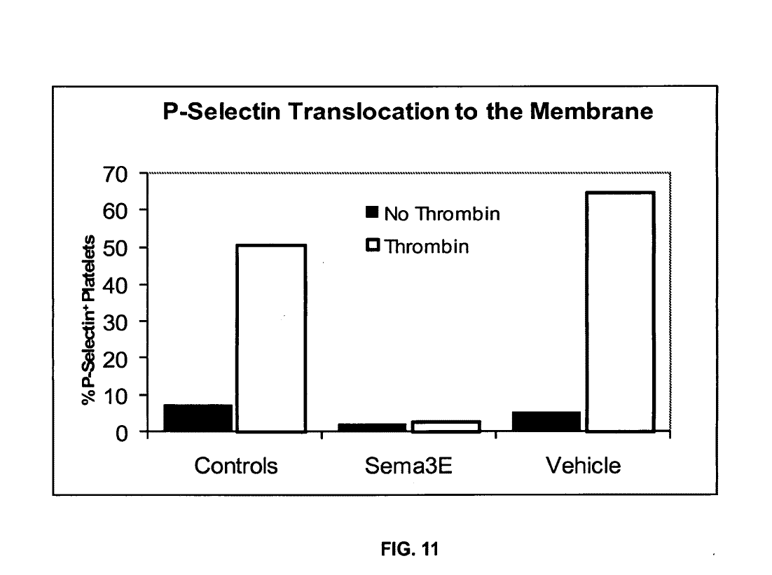

Granular secretion as detected by membrane expression of P-selectin was evaluated in human platelets in the presence or absence of hSema3E (SEQ ID NO: 1). Washed platelets were left untreated, treated with a vehicle only, or treated with a vehicle including hSema3E at a concentrations of 5 μg/ml and 10 μg/ml for a period of 10 minutes at room temperature. Platelets were then treated with 0.05 U/ml thrombin for 15 minutes in the presence of FITC-CD62P antibody. Membrane P-selectin expression was immediately monitored using flow cytometry. Results from platelets treated with 10 μg/ml hSema3E are provided in FIG. 11. As can be seen in FIG. 11, the presence of hSema3E inhibited P-selectin translocation to the membrane.

Two additional human Sema3E polypeptides (SEQ. ID. NO. 5 and SEQ. ID. NO. 6) were evaluated in for their ability to inhibit P-Selectin translocation in human and mouse platelets under similar conditions as those described above. Both such Sema3E polypeptides provided effects comparable to those achieved by hSema3E (SEQ. ID. NO. 1) reported herein.

### Example 9

### Sema3E Inhibits P-Selectin Translocation in Mouse Platelets

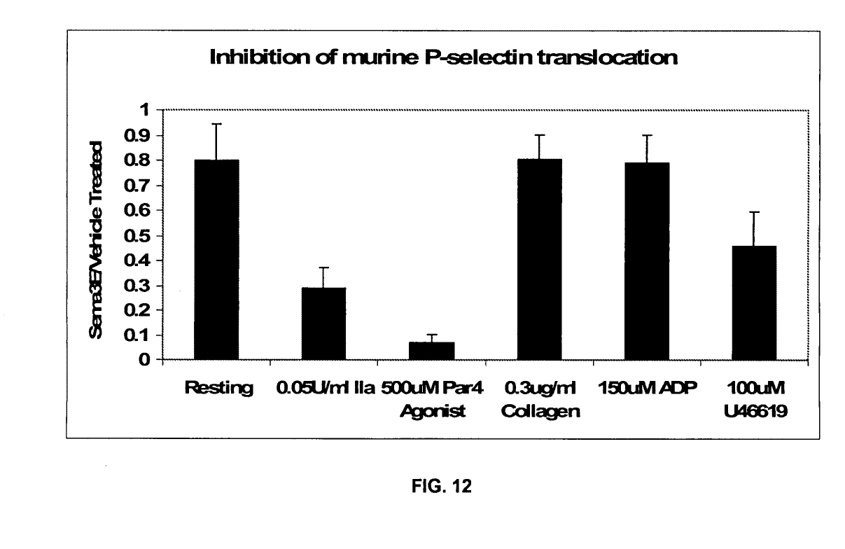

Granular secretion as detected by membrane expression of P-selectin was evaluated in mouse platelets in the presence or absence of Sema3E (recombinant murine semaphorin 3E/Fc) (SEQ ID NO: 2). Washed platelets were treated with a vehicle only, a vehicle including Sema3E at a concentration of 10 μg/ml, or a vehicle including Sema3E at a concentration of 5 μg/ml for a period of 10 minutes at room temperature. Platelets were then treated with one of 0.05 U/ml thrombin (IIa), 500 uM Par4 Agonist, 0.3 μg/ml collagen, 150 uM ADP, and 100 uM U46619, a thromboxane A 2 agonist, for 15 minutes in the presence of FITC-CD62P antibody. Membrane P-selectin expression was immediately monitored using flow cytometry. The results achieved are illustrated in FIG. 12, which shows inhibition achieved in platelets treated with at 10 μg/ml Sema3E.

### Example 10

### Sema3E Inhibits Platelet Aggregation

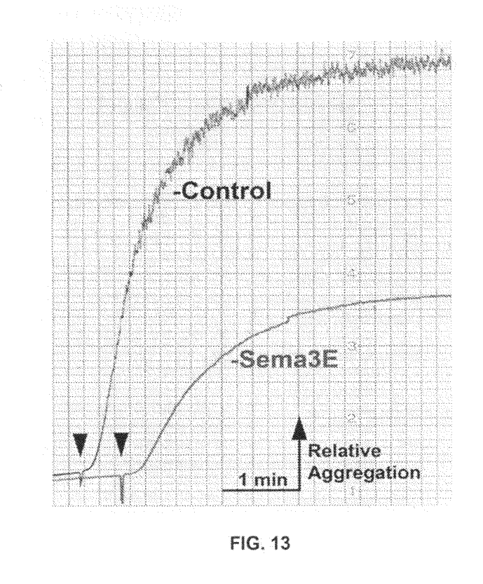

Platelet aggregation was monitored using a platelet aggregometer at 37° C. with a stirring rate at 1000 rpm. Platelets treated either with hSema3E or vehicle (control) were suspended in M199 at 1×10⁹/ml and aggregation was initiated by addition of 0.05 U/ml thrombin. FIG. 13 provides the results of this aggregation study, illustrating that hSema3E (SEQ ID NO: 1) inhibited platelet aggregation.

### Example 11

### Sema3E Inactivates α_{IIb}β₃ Integrin

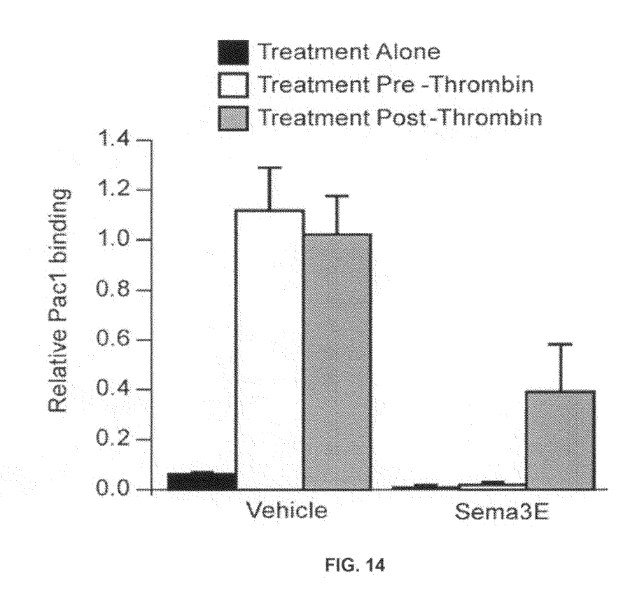

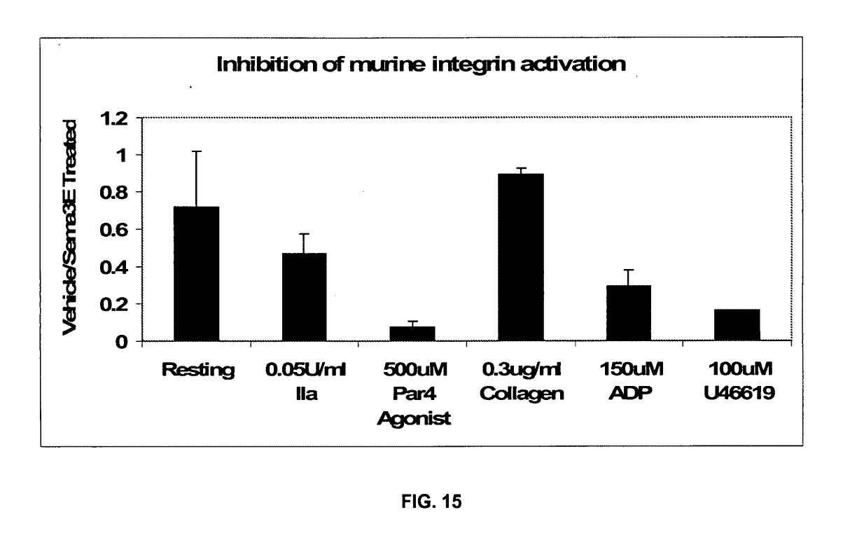

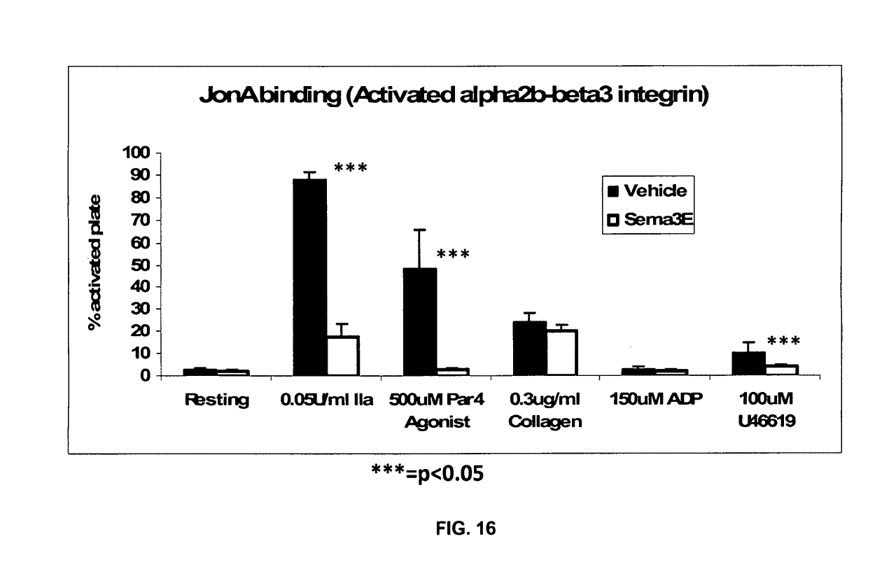

In separate experiments, activation state of α_{IIb}β₃ integrin was monitored for both human and mouse platelets by binding of ligand-mimetic antibodies. In both experiments 1×10⁶ platelets in M199 were preincubated with Sema3E for 10 minutes (hSema3E (SEQ ID NO: 1) for human platelets and recombinant murine semaphorin 3E/Fc (SEQ ID NO: 2) for mouse platelets), followed by incubation with agonists and FITC-conjugated PAC-1 (BD Biosciences, Franklin Lakes, N.J.) for human platelets or PE-JonA (Emfret, Eibelstadt, Germany) for mouse platelets for 15 minutes at room temperature. As represented in FIG. 14, human platelets were incubated with 0.05 U/ml thrombin, while mouse platelets (results shown in FIG. 15 and FIG. 16) were incubated with one of 0.05 U/ml thrombin (IIa), 500 uM Par4 Agonist, 0.3 μg/ml collagen, 150 uM ADP, and 100 uM U46619, a thromboxane A 2 agonist. Platelets were then analyzed immediately on flow cytometry.

The results of these experiments, as illustrated in FIG. 14 through FIG. 16, further demonstrate that Sema3E has the ability to inactivate the α_{IIb}β₃ receptor (as evidenced by the decrease in binding of an activation specific antibody upon treatment with Sema3E, even after treatment with thrombin). Moreover, as evidenced by the information plotted in the grey bar in the vehicle condition shown in FIG. 14, the platelets were activated after being treated with thrombin but when Sema3E was added after thrombin exposure and activation, the platelets no longer bind the FITC-conjugated PAC-1 antibody demonstrating that Sema3E has the ability to reverse or inactivate previously activated platelets, not just inhibit platelets from being activated in the first place. The data generated in these experiments demonstrate that Sema3E has the ability to reverse the activation state of the α_{IIb}β₃ integrin. Moreover, the data provided by these experiments highlight that Sema3E has the capability to inhibit platelet function moderated by several different agonists.

Two additional human Sema3E polypeptides (SEQ. ID. NO. 5 and SEQ. ID. NO. 6) were evaluated in for their ability to inactivate α_{IIb}β₃ integrin under similar conditions as those described above in both human and mouse platelets. Both such Sema3E polypeptides provided effects comparable to those achieved by hSema3E (SEQ. ID. NO. 1) reported herein.

### Example 12

### Sema3E Inhibits Thrombosis In-Vivo

Sema3E was shown to prevent carotid artery occlusion in an in-vivo model of arterial thrombosis. C57BL/6 mice were anesthetized with Avertin (0.3 ml of 2.5%) and anesthesia was maintained using 2% Isoflurane in an isoflurane vaporizer. The skin on top of the throat was removed and the fascia was bluntly dissected to isolate the left jugular vein. A cannula was then inserted into and immobilized to the left jugular vein for administration of Sema3E or saline. The right common carotid artery was exposed. Carotid blood flow was monitored using ultrasound biomicroscopy (UBS, Visualsonic Vevo 660) with a 40 MHz transducer and image-guided 23 MHz spectral pulsed-wave (PW) Doppler. Heart rate, blood flow velocities and blood flow volumes were determined from pulsed Doppler waveforms. During scanning, mouse body temperature was maintained within normal range. Following baseline carotid blood flow readings, 25 g mice were injected with 200 μl of saline, 200 μl saline with 100 ug human Sema3E (SEQ ID NO: 1), 200 U/kg heparin, 50 U/kg heparin, or 10 U/kg heparin 2 minutes prior to FeCl₃ treatment. Thrombosis was induced by applying two pieces of filter paper (1×2 mm) saturated with various concentrations of FeCl₃. The pieces of filter paper were placed on opposite sides of the carotid artery in contact with the adventitial surface of the artery. The FeCl₃ saturated filter paper was removed after 3 minutes and the area was washed 3× with 1 ml saline irrigation. Blood flow was then monitored at 5, 6, 7, 8, 9, 10, 11, 12, 13, 14, 15, 20, 25, and 30 minutes after FeCl₃ application.

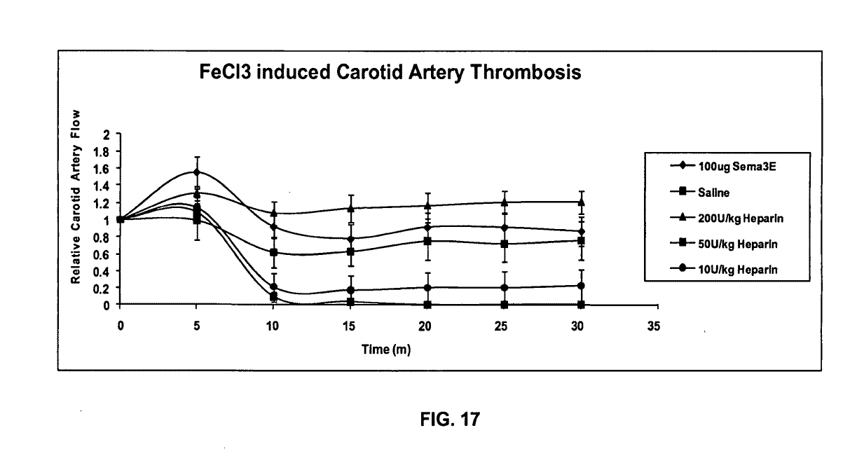

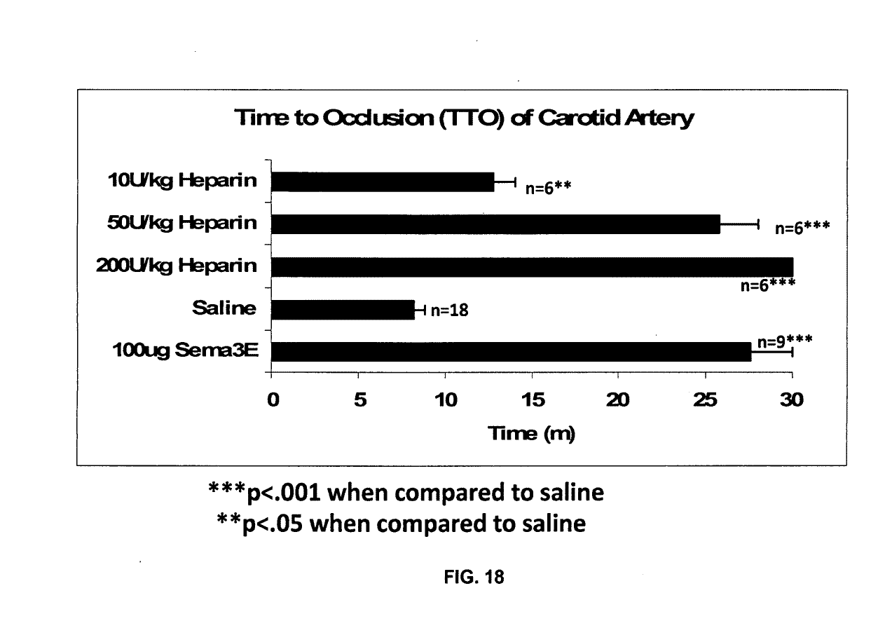

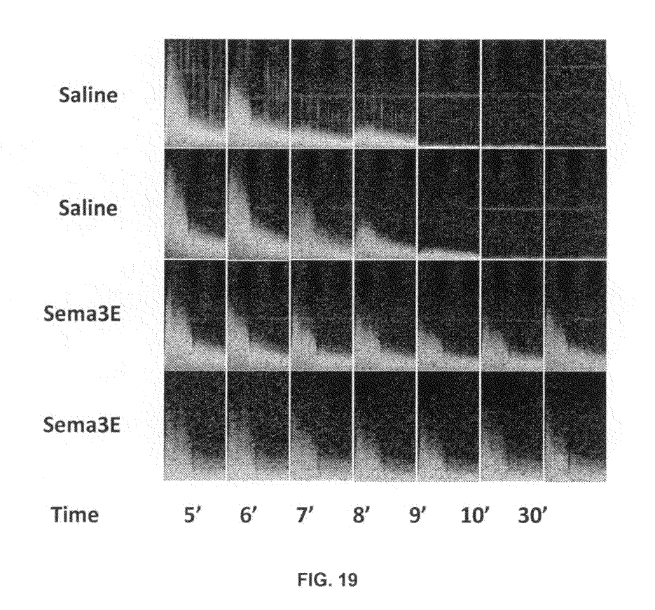

FIG. 17 through FIG. 19 present the results of this in-vivo study. FIG. 17 illustrates the relative flow through the carotid artery for mice receiving the saline, mice receiving saline with Sema3E, and mice receiving varying doses of heparin. FIG. 18 illustrates the time to occlusion achieved in mice receiving the saline, mice receiving saline with Sema3E, and mice receiving the varying doses of heparin. The percent baseline flow results shown in FIG. 17 indicates that blood flow was substantially maintained in Sema3E treated mice, while the time to occlusion data shown in FIG. 18 illustrate that, overall, Sema3E treated mice did not experience occlusions as did mice treated only with Saline. Finally, FIG. 19 illustrates the Doppler flow measured in two examples of mice receiving saline alone and two examples of Sema3E treated mice. The Doppler flow measured in these four examples illustrates blood flow measured as arteries occluded (in the case of saline) or did not occlude (in the case of Sema3E).

### Example 13

### Sema3E Inhibits Activation of Rap1b

Rap1b activation is a common feature of platelet activation via various different agonists. (See, e.g., Wei, A. et al. New Insights into the hemostatic function of platelets. Br J Heamatol. 2009 Jul. 28; Chrzanowska-Wodnicka, M. et al. Rap1b is required for normal platelet function and hemostasis in mice. J. Clin. Invest. 115: 680-687 (2005)). A critical step in platelet activation regulated by Rap1b is α_{IIb}β₃ integrin activation, and Rap1b deficiency impairs soluble fibrinogen binding induced by multiple agonists, indicating that Rap1b is involved upstream from α_{IIb}β₃ integrin activation. Rap1b function is required for the normal α_{IIb}β₃ integrin signaling, and a loss of Rap1b function has been associated with protection against thrombosis. (See, e.g., Wei, A. et al. New Insights into the hemostatic function of platelets. Br J Heamatol. 2009 Jul. 28; Chrzanowska-Wodnicka, M. et al. Rap1b is required for normal platelet function and hemostasis in mice. J. Clin. Invest. 115: 680-687 (2005)).

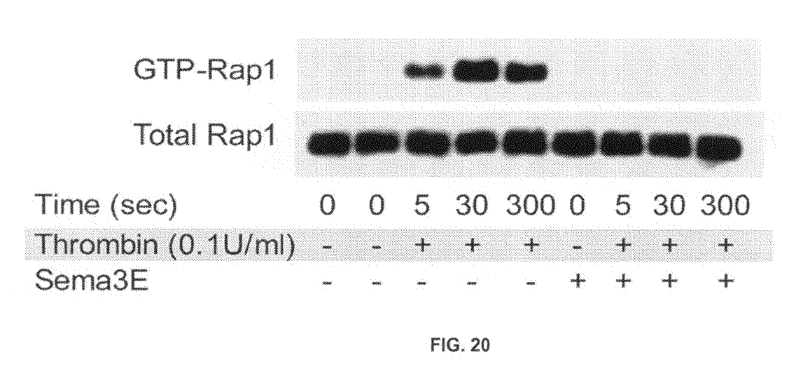

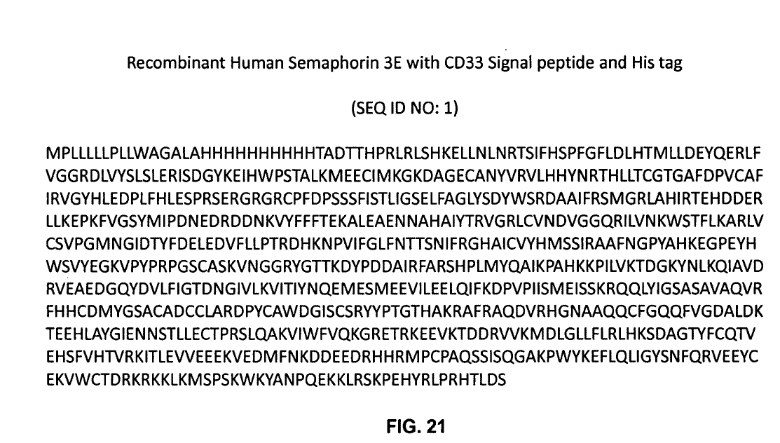

In order to further understand the role of Sema3E in inhibiting platelet function, Rap1b activation in the presence of Sema3E was assessed. Activity of Rap1b was measured using activation assay kits (Upstate) according to the manufacturer's instructions. Briefly, human platelets were treated with 10 ug/ml hSema3E (SEQ ID NO: 1) and/or 0.1 U/ml thrombin as indicated and then lysed in Mg2+ lysis buffer supplemented with protease inhibitors (purchased from Roche). A small portion of the lysate was retained as total cell lysate and the rest was incubated with the assay reagent. GTP-bound forms were eluted from the assay reagent using Laemmli sample buffer and analyzed by western blotting. The total cell lysate was analyzed by blotting for total GTPase input. As can be seen in FIG. 20, in the absence of Sema3E, exposure of the platelets to thrombin caused a rapid increase in GTP Rap1b, with measurable amounts of GTP-Rap1b seen in as little as five seconds after exposure to thrombin and appearing to peak at approximately 30 seconds post-exposure. However, when platelets were exposed to both thrombin and Sema3E, GTP-Rap1b was not detected, demonstrating that Sema3E inhibits activation of Rap1b associated with the presence of thrombin.

### Materials & Methods

### Human Platelet Isolation

Human blood was drawn into acid-citrate-dextrose (ACD) (7 ml ACD/42 ml of blood) and was centrifuged (200 g for 20 min) to obtain platelet-rich plasma (PRP). PRP was re-centrifuged (500 g for 20 min) in the presence of 100 nM PGE-1. The supernatant was discarded and 50 ml of Pipes/saline/glucose (PSG; 5 mM Pipes, 145 mM NaCl, 4 mM KCl, 50 mM Na₂HPO₄, 1 mM MgCl₂-6H2O, and 5.5 mM glucose), containing 100 nM of PGE-1, was used to resuspend the platelet pellet. The platelet suspension was centrifuged (500 g for 20 min), the supernatant was discarded, and the platelet pellet was resuspended in Ca2⁺- and Mg2⁺-free HBSS. All studies are approved by the University of Utah's Institutional Review Board (IRB). The cells were resuspended in medium 199 (M199) at 37° C. for each experiment. Where indicated, the washed platelets were pretreated with Semaphorin3E (10 μg/ml) or vehicle (M199+0.1% human serum albumin) for 10 minutes at room temperature (RT) prior to start of each study

### Mouse Platelet Isolation

Mouse blood was drawn via carotid artery cannula into ACD (150 μl/1 ml blood) Blood/ACD was then diluted 1:2 with PSG and centrifuged (200 g for 10 min) to obtain PRP. PRP diluted again 1:2 with PSG and was re-centrifuged (500 g for 7 min) in the presence of 100 nM PGE-1. The supernatant was discarded and 2 ml of PSG containing 100 nM of PGE-1, was used to resuspend the platelet pellet. The platelet suspension was centrifuged (500 g for 7 min), the supernatant was discarded, and the platelet pellet was resuspended in Ca2⁺- and Mg2⁺-free HBSS.

1. A composition for inhibiting platelet function, the composition comprising a Sema3E polypeptide.

2. A composition according to claim 1, wherein the Sema3E polypeptide is a mammalian Sema3E polypeptide.

3. A composition according to any preceding claim, wherein the Sema3E polypeptide is selected from a human Sema3E polypeptide and a mouse Sema3E polypeptide.

4. A composition according to any preceding claim, wherein the Sema3E polypeptide is selected from an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6.

5. A composition according to claim 4, wherein the Sema3E polypeptide is selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring mammalian Sema3E, or to SEQ. ID. NO. 1, SEQ. ID. NO. 2, SEQ. ID. NO. 5, or SEQ. ID. NO. 6.

6. A composition according to claim 4, wherein the Sema3E polypeptide is selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6., wherein said analog, homolog, or derivative exhibits a polypeptide sequence homology selected from one of 80% or less, 70% or less, 60% or less, or 50% or less to the relevant full-length, naturally occurring human or mouse Sema3E, or to SEQ. ID. NO. 1, SEQ. ID. NO. 2, SEQ. ID. NO. 5, or SEQ. ID. NO. 6.

7. A composition according to any preceding claim wherein the Sema3E polypeptide inhibits one or more of platelet spreading, adhesion, aggregation, α-granule release and clotting.

8. A composition according to any preceding claim, wherein the Sema3E polypeptide inhibits each of platelet spreading, adhesion, aggregation, α-granule release and clotting.

9. A composition according to any of claims 1 through 6, wherein the Sema3E polypeptide reverses one or more of platelet spreading, adhesion, aggregation, and clotting.

10. A composition according to any of claims 1 through 6 and 9, wherein the Sema3E polypeptide reverses each of more of platelet spreading, adhesion, aggregation, and clotting.

11. A composition according to any preceding claim, wherein the platelets are human platelets.

12. A composition according to any preceding claim, wherein the composition is a pharmaceutical formulation and further comprises a pharmaceutically acceptable carrier.

13. A composition according to claim 12, wherein the pharmaceutical formulation further comprises one or more pharmaceutically acceptable excipients.

14. A composition according to any preceding claim, wherein the Sema3E polypeptide inhibits activation of Rap1b.

15. A composition according to any preceding claim, wherein the Sema3E polypeptide binds to and is biologically active at a plexin receptor.

16. A composition according to claim 15, wherein the Sema3E binds to and is active at a PlexinD1 receptor.

17. A composition according to claim 15, wherein the Sema3E binds to and is active at a PlexinD1 receptor and a Plexin A receptor selected from Plexin A1 through Plexin A4.

18. A method for inhibiting the function of platelet cells, the method comprising providing a Sema3E polypeptide and exposing said platelet cells to said Sema3E polypeptide.

19. A method according to claim 18, wherein providing a Sema3E polypeptide comprises providing a mammalian Sema3E polypeptide.

20. A method according to either of claim 18 or 19, wherein providing a Sema3E polypeptide comprises providing a polypeptide selected from a human Sema3E polypeptide and a mouse Sema3E polypeptide.

21. A method according to any of claims 18 through 20, wherein providing a Sema3E polypeptide comprises providing a polypeptide selected from an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6.

22. A method according to any of claims 18 through 21, wherein providing a Sema3E polypeptide comprises providing a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring mammalian Sema3E, or to SEQ. ID. NO. 1, SEQ. ID. NO. 2, SEQ. ID. NO. 5, or SEQ. ID. NO. 6.

23. A method according to any of claims 18 through 21, wherein providing a Sema3E polypeptide comprises providing a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology selected from one of 80% or less, 70% or less, 60% or less, or 50% or less to the relevant full-length, naturally occurring mammalian Sema3E, or to SEQ. ID. NO. 1, SEQ. ID. NO. 2, SEQ. ID. NO. 5, or SEQ. ID. NO. 6.

24. A method according to any of claims 18 through 23, wherein exposing said platelet cells to said Sema3E polypeptide inhibits one or more of platelet spreading, adhesion, aggregation, α-granule release and clotting.

25. A method according to any of claims 18 through 24, wherein exposing said platelet cells to said Sema3E polypeptide inhibits each of platelet spreading, adhesion, aggregation, α-granule release and clotting.

26. A method according to any of claims 18 through 25, wherein exposing said platelet cells to said Sema3E polypeptide, comprises exposing human platelet cells to said Sema3E polypeptide.

27. A method according to any of claims 18 through 26, wherein providing a Sema3E polypeptide comprises providing a pharmaceutical formulation comprising a Sema3E polypeptide and a pharmaceutically acceptable carrier.

28. A method according to claim 27, wherein the pharmaceutical formulation comprising a Sema3E polypeptide further comprises one or more pharmaceutically acceptable excipients.

29. A method according to any of claims 18 through 28, wherein providing a Sema3E polypeptide comprises providing a Sema3E polypeptide that binds to and is biologically active at a plexin receptor.

30. A method according to any of claims 18 through 29, wherein providing a Sema3E polypeptide comprises providing a Sema3E polypeptide that binds to and is biologically active at a PlexinD1 receptor.

31. A method according to any of claims 18 through 30, wherein providing a Sema3E polypeptide comprises providing a Sema3E polypeptide that binds to and is biologically active at a PlexinD1 receptor and a Plexin A receptor selected from Plexin A1 through Plexin A4.

32. A method according to any of claims 18 through 31, wherein exposing said platelet cells to said Sema3E polypeptide results in inhibition of activation of Rap1b.

33. A method for reversing the function of platelet cells, the method comprising providing a Sema3E polypeptide and exposing said platelet cells to said Sema3E polypeptide.

34. A method according to claim 33, wherein providing a Sema3E polypeptide comprises providing a mammalian Sema3E polypeptide.

35. A method according to either of claim 33 or 34, wherein providing a Sema3E polypeptide comprises providing a polypeptide selected from a human Sema3E polypeptide and a mouse Sema3E polypeptide.

36. A method according to any of claims 33 through 35, wherein providing a Sema3E polypeptide comprises providing a polypeptide selected from an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6.

37. A method according to any of claims 33 through 36, wherein providing a Sema3E polypeptide comprises providing a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring mammalian Sema3E, or to SEQ. ID. NO. 1, SEQ. ID. NO. 2, SEQ. ID. NO. 5, or SEQ. ID. NO. 6.

38. A method according to any of claims 33 through 36, wherein providing a Sema3E polypeptide comprises providing a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology selected from one of 80% or less, 70% or less, 60% or less, or 50% or less to the relevant full-length, naturally occurring mammalian Sema3E, or to SEQ. ID. NO. 1, SEQ. ID. NO. 2, SEQ. ID. NO. 5, or SEQ. ID. NO. 6.

39. A method according to any of claims 33 through 38, wherein exposing said platelet cells to said Sema3E polypeptide reverses one or more of platelet spreading, adhesion, aggregation, and clotting.

40. A method according to any of claims 33 through 39, wherein exposing said platelet cells to said Sema3E polypeptide reverses each of platelet spreading, adhesion, aggregation, and clotting.

41. A method according to any of claims 33 through 40, wherein exposing said platelet cells to said Sema3E polypeptide, comprises exposing human platelet cells to said Sema3E polypeptide.

42. A method according to any of claims 33 through 41, wherein providing a Sema3E polypeptide comprises providing a pharmaceutical formulation comprising a Sema3E polypeptide and a pharmaceutically acceptable carrier.

43. A method according to claim 42, wherein the pharmaceutical formulation comprising a Sema3E polypeptide further comprises one or more pharmaceutically acceptable excipients.

44. A method according to any of claims 33 through 43, wherein providing a Sema3E polypeptide comprises providing a Sema3E polypeptide that binds to and is biologically active at a plexin receptor.

45. A method according to any of claims 33 through 44, wherein providing a Sema3E polypeptide comprises providing a Sema3E polypeptide that binds to and is biologically active at a PlexinD1 receptor.

46. A method according to any of claims 33 through 45, wherein providing a Sema3E polypeptide comprises providing a Sema3E polypeptide that binds to and is biologically active at a PlexinD1 receptor and a Plexin A receptor selected from Plexin A1 through Plexin A4.

47. A method according to any of claims 33 through 46, wherein exposing said platelet cells to said Sema3E polypeptide results in inhibition of activation of Rap1b.

48. A method for treating a patient at risk for a pathologic condition associated with one or more platelet function, the method comprising identifying a patient at risk for a pathologic condition associated with one or more of platelet spreading, adhesion, aggregation, α-granule release and clotting and administering a therapeutically effective amount of a Sema3E polypeptide to said patient.

49. A method according to claim 48, wherein the pathologic condition is selected from stroke, myocardial infarction, unstable angina, angina, thrombosis, including deep venous thrombosis, pulmonary embolism, and atrial fibrillation.

50. A method according to either of claims 48 and 49, wherein administering a therapeutically effective amount of a Sema3E polypeptide to said patient comprises administering a mammalian Sema3E polypeptide.

51. A method according to any of claims 48 through 50, wherein administering a therapeutically effective amount of a Sema3E polypeptide to said patient comprises administering a polypeptide selected from a human Sema3E polypeptide and a mouse Sema3E polypeptide.

52. A method according to any of claims 48 through 51, wherein administering a therapeutically effective amount of a Sema3E polypeptide to said patient comprises administering a polypeptide selected from an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6.

53. A method according to any of claims 48 through 52, wherein administering a therapeutically effective amount of a Sema3E polypeptide to said patient comprises administering a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring mammalian Sema3E, or to SEQ. ID. NO. 1, SEQ. ID. NO. 2, SEQ. ID. NO. 5, or SEQ. ID. NO. 6.

54. A method according to any of claims 48 through 52, wherein administering a therapeutically effective amount of a Sema3E polypeptide to said patient comprises administering a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology selected from one of 80% or less, 70% or less, 60% or less, or 50% or less to the relevant full-length, naturally occurring mammalian Sema3E, or to SEQ. ID. NO. 1, SEQ. ID. NO. 2, SEQ. ID. NO. 5, or SEQ. ID. NO. 6.

55. A method according to any of claims 48 through 54, wherein administering a therapeutically effective amount of a Sema3E polypeptide to said patient inhibits one or more of platelet spreading, adhesion, aggregation, α-granule release and clotting.

56. A method according to any of claims 48 through 55, wherein administering a therapeutically effective amount of a Sema3E polypeptide to said patient inhibits each of platelet spreading, adhesion, aggregation, α-granule release and clotting.

57. A method according to any of claims 48 through 56, wherein administering a therapeutically effective amount of a Sema3E polypeptide to said patient comprises administering a pharmaceutical formulation comprising a Sema3E polypeptide and a pharmaceutically acceptable carrier.

58. A method according to claim 57, wherein the pharmaceutical formulation comprising a Sema3E polypeptide further comprises one or more pharmaceutically acceptable excipients.

59. A method according to any of claims 48 through 58, wherein administering a therapeutically effective amount of a Sema3E polypeptide to said patient comprises administering a Sema3E polypeptide that binds to and is biologically active at a plexin receptor.

60. A method according to any of claims 48 through 59, wherein administering a therapeutically effective amount of a Sema3E polypeptide to said patient comprises administering a Sema3E polypeptide that binds to and is biologically active at a PlexinD1 receptor.

61. A method according to any of claims 48 through 60, wherein administering a therapeutically effective amount of a Sema3E polypeptide to said patient comprises administering a Sema3E polypeptide that binds to and is biologically active at a PlexinD1 receptor and a Plexin A receptor selected from Plexin A1 through Plexin A4.

62. A method according to any of claims 48 through 61, wherein administering a therapeutically effective amount of a Sema3E polypeptide to said patient results in inhibition of activation of Rap1b in one or more platelet cells.

63. A method for treating a patient suffering from a pathologic condition associated with one or more platelet function, the method comprising identifying a patient suffering from a pathologic condition associated with one or more of platelet spreading, adhesion, aggregation, α-granule release and clotting and administering a therapeutically effective amount of a Sema3E polypeptide to said patient.

64. A method according to claim 63, wherein the pathologic condition is selected from stroke, myocardial infarction, unstable angina, angina, thrombosis, including deep venous thrombosis, pulmonary embolism, and atrial fibrillation.

65. A method according to either of claims 63 and 64, wherein administering a therapeutically effective amount of a Sema3E polypeptide to said patient comprises administering a mammalian Sema3E polypeptide.

66. A method according to any of claims 63 through 65, wherein administering a therapeutically effective amount of a Sema3E polypeptide to said patient comprises administering a polypeptide selected from a human Sema3E polypeptide and a mouse Sema3E polypeptide.

67. A method according to any of claims 63 through 66, wherein administering a therapeutically effective amount of a Sema3E polypeptide to said patient comprises administering a polypeptide selected from an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6.

68. A method according to any of claims 63 through 67, wherein administering a therapeutically effective amount of a Sema3E polypeptide to said patient comprises administering a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology of at least about 80%, 81%, 82%, 83%, 84%, 85%, 86%, 87%, 88%, 89%, 90%, 91%, 92%, 93%, 94%, 95%, 96%, 97%, 98%, 99% or greater to the relevant full-length, naturally occurring mammalian Sema3E, or SEQ. ID. NO. 1, SEQ. ID. NO. 2, SEQ. ID. NO. 5, or SEQ. ID. NO. 6.

69. A method according to any of claims 63 through 67, wherein administering a therapeutically effective amount of a Sema3E polypeptide to said patient comprises administering a polypeptide selected from an analog, homolog, or derivative of an isolated and purified, full-length, naturally occurring mammalian Sema3E polypeptide, a Sema3E polypeptide according to SEQ. ID. NO. 1, a Sema3E polypeptide according to SEQ. ID. NO. 2, a Sema3E polypeptide according to SEQ. ID. NO. 5, and a Sema3E polypeptide according to SEQ. ID. NO. 6, wherein said analog, homolog, or derivative exhibits a polypeptide sequence homology selected from one of 80% or less, 70% or less, 60% or less, or 50% or less to the relevant full-length, naturally occurring mammalian Sema3E, or to SEQ. ID. NO. 1, SEQ. ID. NO. 2, SEQ. ID. NO. 5, or SEQ. ID. NO. 6.

70. A method according to any of claims 63 through 69, wherein administering a therapeutically effective amount of a Sema3E polypeptide to said patient inhibits one or more of platelet spreading, adhesion, aggregation, α-granule release and clotting.

71. A method according to any of claims 63 through 70, wherein administering a therapeutically effective amount of a Sema3E polypeptide to said patient inhibits each of platelet spreading, adhesion, aggregation, α-granule release and clotting.

72. A method according to any of claims 63 through 69, wherein administering a therapeutically effective amount of a Sema3E polypeptide to said patient reverses one or more of platelet spreading, adhesion, aggregation, and clotting.

73. A method according to any of claims 63 through 69, wherein administering a therapeutically effective amount of a Sema3E polypeptide to said patient reverses each of platelet spreading, adhesion, aggregation, and clotting.

74. A method according to any of claims 63 through 73, wherein administering a therapeutically effective amount of a Sema3E polypeptide to said patient comprises administering a pharmaceutical formulation comprising a Sema3E polypeptide and a pharmaceutically acceptable carrier.

75. A method according to claim 74, wherein the pharmaceutical formulation comprising a Sema3E polypeptide further comprises one or more pharmaceutically acceptable excipients.

76. A method according to any of claims 63 through 75, wherein administering a therapeutically effective amount of a Sema3E polypeptide to said patient comprises administering a Sema3E polypeptide that binds to and is biologically active at a plexin receptor.

77. A method according to any of claims 63 through 76, wherein administering a therapeutically effective amount of a Sema3E polypeptide to said patient comprises administering a Sema3E polypeptide that binds to and is biologically active at a PlexinD1 receptor.

78. A method according to any of claims 63 through 77, wherein administering a therapeutically effective amount of a Sema3E polypeptide to said patient comprises administering a Sema3E polypeptide that binds to and is biologically active at a PlexinD1 receptor and a Plexin A receptor selected from Plexin A1 through Plexin A4.

79. A method according to any of claims 63 through 78, wherein administering a therapeutically effective amount of a Sema3E polypeptide to said patient results in inhibition of activation of Rap1b in one or more platelet cells.


SEQUENCE LISTING (6 sequences)

SEQ ID NO: 1 - protein, 777 aa, Homo sapiens
MPLLLLLPLLWAGALAHHHHHHHHHHTADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRAFRAQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 2 - protein, 993 aa, Homo sapiens
MPLLLLLPLLWAGALANPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRAFRAQDVRHGNAAQQCFGQQFVGDALDRTEERLAYGIESNSTLLECTPRSLQAKVIWFVQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHFIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 1007 aa, Homo sapiens [sequence omitted]

SEQ ID NO: 4 - protein, 989 aa, Homo sapiens [sequence omitted]

SEQ ID NO: 5 - protein, 757 aa, Homo sapiens
HHHHHHHHNPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKRRFRRQDVRHGNAAQQCFGQQFVGDALDRTEERLAYGIESNSTLLECTPRSLQAKVIWFVQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHFRLPRHTLLS

SEQ ID NO: 6 - protein, 757 aa, Homo sapiens
HHHHHHHHNPSYPRLRLSHKELLELNRTSIFQSPLGFLDLHTMLLDEYQERLFVGGRDLVYSLNLERVSDGYREIYWPSTAVKVEECIMKGKDANECANYIRVLHHYNRTHLLTCATGAFDPHCAFIRVGHHSEEPLFHLESHRSERGRGRCPFDPNSSFVSTLVGNELFAGLYSDYWGRDSAIFRSMGKLGHIRTEHDDERLLKEPKFVGSYMIPDNEDRDDNKMYFFFTEKALEAENNAHTIYTRVGRLCVNDMGGQRILVNKWSTFLKARLVCSVPGMNGIDTYFDELEDVFLLPTRDPKNPVIFGLFNTTSNIFRGHAVCVYHMSSIREAFNGPYAHKEGPEYHWSLYEGKVPYPRPGSCASKVNGGKYGTTKDYPDDAIRFARMHPLMYQPIKPVHKKPILVKTDGKYNLRQLAVDRVEAEDGQYDVLFIGTDTGIVLKVITIYNQETEWMEEVILEELQIFKDPAPIISMEISSKRQQLYIGSASAVAQVRFHHCDMYGSACADCCLARDPYCAWDGISCSRYYPTGAHAKARFARQDVRHGNAAQQCFGQQFVGDALDRTEERLAYGIESNSTLLECTPRSLQAKVIWFVQKGRDVRKEEVKTDDRVVKMDLGLLFLRVRKSDAGTYFCQTVEHNFVHTVRKITLEVVEEHKVEGMFHKDHEEERHHKMPCPPLSGMSQGTKPWYKEFLQLIGYSNFQRVEEYCEKVWCTDKKRKKLKMSPSKWKYANPQEKRLRSKAEHFRLPRHTLLS